(19) United States
(12) Patent Application Publication
(10) Pub. No.: US 2025/0262246 A1
(43) Pub. Date: Aug. 21, 2025

(54) CD83+, CD83+PD-L1+ MESENCHYMAL STEM CELLS AND PREPARATION METHODS AND USE THEREOF

(71) Applicant: CHINESE PEOPLE'S LIBERATION ARMY SPECIAL MEDICAL CENTER, Chongqing (CN)

(72) Inventors: Xiang XU, Chongqing (CN); Xuewei XIA, Chongqing (CN); Mengwei YAO, Chongqing (CN); Zhan LI, Chongqing (CN); Wei XING, Chongqing (CN)

(73) Assignee: CHINESE PEOPLE'S LIBERATION ARMY SPECIAL MEDICAL CENTER, Chongqing (CN)

(21) Appl. No.: 19/199,381

(22) Filed: May 5, 2025

Related U.S. Application Data
(63) Continuation of application No. PCT/CN2023/135087, filed on Nov. 29, 2023.

(30) Foreign Application Priority Data
Nov. 21, 2023 (CN) 202311550534.7

(51) Int. Cl. A61K 35/28; A61P 29/00; A61P 37/06; C12N 5/0775

(52) U.S. Cl. CPC A61K 35/28; A61P 29/00; A61P 37/06; C12N 5/0668; C12N 2501/24; C12N 2501/25

(57) ABSTRACT

Use of CD83⁺ mesenchymal stem cells/CD83⁺ overexpressed mesenchymal stem cells in the preparation of drugs for preventing/treating at least one of autoimmune disease or inflammatory-related disease is provided. The CD83⁺ mesenchymal stem cells/CD83⁺ overexpressed mesenchymal stem cells are capable of increasing the secretion of at least one of anti-inflammatory factor or immunosuppressive factor. Immunosuppressive or anti-inflammatory function-prone CD83⁺ mesenchymal stem cells/CD83⁺ overexpressed mesenchymal stem cells and a preparation method for immunosuppressive or anti-inflammatory function enhanced CD83⁺ PD-L1⁺ mesenchymal stem cells are also provided. The immunosuppressive or anti-inflammatory function enhanced CD83⁺PD-L1⁺-MSCs can significantly reduce the mortality rate and inflammatory response of the sepsis in the treatment of sepsis and can also significantly improve the clinical symptom of the arthritis in mice in the treatment of collagen induced arthritis. Anti-inflammatory CD83⁺PD-L1⁺-MSCs induction kit can be further developed based on an induction process of anti-inflammatory CD83⁺PD-L1⁺-MSCs, which can be readily popularized for application.

### CROSS-REFERENCE TO RELATED APPLICATIONS

The present application is a Continuation of International Application No. PCT/CN2023/135087, filed on Nov. 29, 2023, which claims priority to Chinese Patent Application No. 202311550534.7, filed on Nov. 21, 2023, the entire contents of each of which are incorporated herein by reference.

### TECHNICAL FIELD

The present disclosure relates to the field of biomedical technology, and in particular, to immunosuppressive or anti-inflammatory function-prone CD83⁺ mesenchymal stem cells, and immunosuppressive or anti-inflammatory function enhanced CD83⁺PD-L1⁺ mesenchymal stem cells, and an in vitro screening or gene modification overexpression kit, an immunosuppressive or anti-inflammatory induction kit, a quality assessment method and use thereof.

### BACKGROUND

With the intensive research on stem cell therapy techniques, Mesenchymal Stem Cells (MSCs) have been recognized as an ideal tool for the treatment of tissue-damaging diseases and immune or inflammation related diseases. The MSCs are a kind of pluripotent stem cells with self-renewal and multidirectional differentiation potential, and the MSCs are widely distributed in tissues such as adult bone marrow, adipose, dental pulp, placenta, and umbilical cord. Numerous studies have confirmed that the MSCs not only have strong self-renewal and multidirectional differentiation potential to mesoderm, ectoderm and endoderm, but also have good immune/inflammatory regulation ability. The MSCs can effectively regulate the imbalance of immune/inflammatory response in the body by regulating the proliferation, differentiation, secretion, and polarization of immune cells such as T cells, NK cells, macrophages, etc. At the same time, the MSCs can effectively inhibit apoptosis of in-situ cells in damaged tissues, promote angiogenesis, activate in-situ stem cells and other biological characteristics, and participate in the regeneration and repair of damaged tissues. Therefore, MSCs transplantation not only restores imbalanced immune response or inflammatory response of a patient, but also has tissue regeneration and repair functions. Based on these characteristics, the MSCs transplantation is expected to be an ideal strategy for the treatment of injury, inflammation, and immune-related diseases.

It has been confirmed that, in addition to directly participating in the regeneration and repair of damaged tissues through differentiation and secretion of cell growth factors, the MSCs can also regulate the imbalance of inflammation/immune response to repair immune homeostasis, avoid secondary inflammatory injury of target organs, improve the microenvironment for tissue repair, and promote the regeneration and repair of damaged tissues. Given that the MSCs transplantation for the treatment of inflammatory and immune-related diseases can fundamentally restore the imbalance of inflammatory/immune response in the body and promote the regeneration and repair of damaged tissues, the MSCs transplantation is considered to be an ideal strategy with great potential for the treatment of injury, inflammation, and immune-related diseases. However, with the increase in the amount of clinical data on the MSCs transplantation in the treatment of inflammatory/immune-related diseases, it has been found that there are huge individual differences in the clinical efficacy of the MSCs transplantation for the treatment of inflammatory/immune-related diseases. Even for the same diseases, the MSCs transplantation can be therapeutic for some patients, but for other patients, it may be ineffective or even has opposite results.

With the in-depth understanding of the characteristics of the MSCs and the inflammatory/immune regulatory mechanisms, it has been found that the MSCs have no tissue repair and inflammatory regulatory activities in their natural state, and their tissue repair and immune regulatory functions are enabled by microenvironmental stimulation. In terms of tissue repair, the MSCs in resting state have no tissue repair ability, and the acquisition of their tissue repair ability depends on the stimulation of growth factors such as TGF-β, BMP, HGF, PDGF, and different stimulus factors can induce different differentiation directions of the MSCs and the secretion spectrum of cell growth factor, thus affecting the tissue repair function of the MSCs, and presenting different regeneration and repair results. Similarly, the inflammatory/immune regulatory function of the MSCs is not constitutive, i.e., the inflammatory/immune regulatory ability of the MSCs is low in the natural state, and the acquisition of the inflammatory/immune regulatory function of the MSCs is dependent on stimulation by inflammatory/immune microenvironment, and regulated by numerous factors, e.g., the MSCs need to be stimulated by inflammatory factors such as IFN-γ, TNF-α, and IL-1β to undergo immunosuppressive or anti-inflammatory reprogramming, and then express anti-inflammatory mediators or receptors such as PD-L1, IDO, and IL-10, in order to exert the immunosuppressive or anti-inflammatory effects. Additionally, the regulation of host inflammation/immunity by the MSCs is bi-directional, including activating immunity or pro-inflammation, and inhibiting immunity or anti-inflammation. The bidirectionality of the inflammatory/immune regulation of the MSCs greatly affects the clinical efficacy of the MSCs in the treatment of inflammatory/immune diseases. It has been demonstrated that TGF-β1 and TLR4 agonist LPS induce the reprogramming of the MSCs to an immune-activating or pro-inflammatory phenotype, exerting pro-inflammatory effects. TLR3 agonists or inflammatory factors (e.g., IFN-γ, IL-1β, TNF-α, etc.) then induce reprogramming of the MSCs to an anti-inflammatory or immunosuppressive phenotype, exerting an inflammation-suppressive effect. However, due to differences in etiology and course of disease of many patients with inflammatory/immune-related diseases, factors or mediators that can induce both pro-inflammatory and anti-inflammatory MSCs often coexist in the microenvironment of some patients, resulting in the co-existence of pro-inflammatory/anti-inflammatory MSCs; or some patients lack anti-inflammatory enabling factors, resulting in anti-inflammatory enabling reprogramming disorder of MSCs. The above reasons may be important reasons for the large individual differences in the therapeutic resistance and clinical efficacy of the MSCs transplantation for the treatment of inflammatory/immune-related diseases. Therefore, it is a key technical link about how to empower and maintain the anti-inflammatory or immunosuppressive function of the MSCs to improve the clinical efficacy of the MSCs transplantation for the treatment of inflammatory/immune-related diseases.

A large count of studies has confirmed that the MSCs are heterogeneous population of multipotential stem cells with multifunctional subpopulations and tissue origin differences. Wang Z et al. used scRNA-seq to compare differences in MSCs from adipose, bone marrow, and umbilical cord and identified seven tissue-specific subpopulations of MSCs and five functionally conserved subpopulations of MSCs, with umbilical cord MSCs (hUC-MSCs) having a stronger inflammatory regulatory potential in comparison. Zhang S et al. identified two functional subpopulations with differential inflammatory regulation and tissue differentiation functions in primary MSCs. However, although there have been studies on the heterogeneity of MSCs, only briefly comparison was made on the differences in their tissue origins, lacking information on the characteristics of the functional subgroups. Additionally, heterogeneity data obtained from static MSCs are difficult to represent information on spatiotemporally enabled programming of inflammatory regulatory subgroups of MSCs during inflammatory disease processes.

### SUMMARY

One or more embodiments of the present disclosure provide a method for preventing/treating of an autoimmune disease or an inflammatory-related disease, comprising: administering a therapeutically effective amount of a pharmaceutical composition comprising CD83⁺ mesenchymal stem cells/CD83⁺ overexpressed mesenchymal stem cells to a subject suffering from the autoimmune disease or the inflammatory-related disease.

In some embodiments, the pharmaceutical composition further comprises CD83⁺PD-L1⁺ mesenchymal stem cells.

One or more embodiments of the present disclosure provide a preparation method for CD83⁺ mesenchymal stem cells/CD83⁺ overexpressed mesenchymal stem cells, comprising: (a) isolating and culturing the mesenchymal stem cells; (b) sorting and obtaining immunosuppressive or anti-inflammatory function-prone natural CD83⁺ MSCs/CD83⁺ overexpressed MSCs by magnetic beads coupled with anti-CD83 antibodies and flow sorting technology, or lentiviral gene transduction technology.

One or more embodiments of the present disclosure provide a preparation method for CD83⁺PD-L1⁺ mesenchymal stem cells, comprising obtaining immunosuppressive or anti-inflammatory function enhanced CD83⁺PD-L1⁺ mesenchymal stem cells by inducing and culturing the CD83⁺ mesenchymal stem cells using an immune factor inducing complex.

One or more embodiments of the present disclosure provide a quality assessment method for the CD83⁺PD-L1⁺ mesenchymal stem cells obtained by the preparation method, a positive expression rate of CD83 and a positive expression rate of PD-L1 receptor in the CD83⁺PD-L1⁺ mesenchymal stem cells are used as quality control indicators of the CD83⁺PD-L1⁺ mesenchymal stem cells.

One or more embodiments of the present disclosure provide a kit, the kit is effectively used for evaluating a quality requirement of the CD83⁺PD-L1⁺ mesenchymal stem cells by detecting the positive expression rate of the CD83 and the positive expression rate of the PD-L1 receptor with the quality assessment method; and the kit further comprises the immune factor inducing complex.

### BRIEF DESCRIPTION OF THE DRAWINGS

In order to more clearly illustrate specific embodiments of the present disclosure, the following is a brief description of the accompanying drawings to be used in the description of specific embodiments or prior art:

FIGS. 1A-1G are diagrams of flow assay results for surface marker molecules of WT-MSCs according to some embodiments of the present disclosure;

FIG. 2 is a diagram of identification results of the triple lineage differentiation of CD83⁻-MSCs, CD83⁺-MSCs, and WT-MSCs according to some embodiments of the present disclosure;

FIGS. 3A-3B are diagrams of flow assay results for positive expression rates of CD83 of pre-sorting MSCs and post-sorting MSCs according to some embodiments of the present disclosure;

FIGS. 4A-4B are diagrams of assay results of human CD83 gene-modified CD83⁺ overexpressed mesenchymal stem cells according to some embodiments of the present disclosure;

FIGS. 5A-5B are diagrams of flow assay results for cell surface marker molecules of CD83⁻-MSCs and CD83⁺-MSCs according to some embodiments of the present disclosure;

FIGS. 6A-6D are diagrams of assay results for the cell proliferative capacity for the CD83⁻-MSCs and CD83⁺-MSCs according to some embodiments of the present disclosure;

FIGS. 7A-7B are diagrams of assay results for cell clonal proliferative capacity of the CD83⁻-MSCs and CD83⁺-MSCs according to some embodiments of the present disclosure;

FIGS. 8A-8D are diagrams of assay results for cell migration capacity of the CD83⁻-MSCs and CD83⁺-MSCs according to some embodiments of the present disclosure;

FIGS. 9A-9H are diagrams of assay evaluation results for in vitro anti-inflammatory function of CD83⁻-MSCs, CD83⁺-MSCs, and WT-MSCs according to some embodiments of the present disclosure;

FIGS. 10A-10D are diagrams of assay results for IDO1 and PD-L1 gene and protein expression in MSCs induced by different concentrations of TNF-α in combination with IFN-γ according to some embodiments of the present disclosure;

FIGS. 11A-11C are diagrams of time-dynamic assay results for PD-L1 expression on membranes of the CD83⁺-MSCs induced by the TNF-α in combination with the IFN-γ according to some embodiments of the present disclosure;

FIGS. 12A-12K are diagrams of evaluation results for the efficacy of CD83⁻-MSCs, CD83⁺-MSCs, and WT-MSCs in the treatment of rheumatoid arthritis according to some embodiments of the present disclosure;

FIGS. 13A-13J are diagrams of evaluation results for the efficacy of natural CD83⁺ or overexpressed artificial CD83⁺ overexpressed mesenchymal stem cells in the treatment of rheumatoid arthritis according to some embodiments of the present disclosure;

FIGS. 14A-14L are diagrams of evaluation results for the efficacy of CD83⁺ or overexpressed artificial CD83⁺ overexpressed MSCs in the treatment of sepsis according to some embodiments of the present disclosure; and

FIGS. 15A-15B are diagrams of histopathological examination results of hematoxylin and eosin (H&E) staining of CLP sepsis mice after 72 h of treatment on CLP sepsis mice with sham control (sham), PBS control, WT-MSCs, CD83⁺-MSCs, CD83^{+/OE}-MSCs and WT^{CD83 OE}-MSCs according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. The present disclosure can be applied to other similar scenarios based on these drawings without creative labor. Unless obviously obtained from the context or the context illustrates otherwise, the same numeral in the drawings refers to the same structure or operation.

As shown in the present disclosure and in the claims, unless the context clearly suggests an exception, the words “a”, “one”, “an” and/or “the” do not refer specifically to the singular but may also include the plural. ″, “a”, “an”, and/or “the” do not refer specifically to the singular, but may also include the plural. Generally, the terms “including” and “comprising” suggest only the inclusion of clearly identified steps and elements. In general, the terms “including” and “comprising” only suggest the inclusion of explicitly identified steps and elements that do not constitute an exclusive list, and the method or apparatus may also include other steps or elements.

Embodiments of the present disclosure provide a method for preventing/treating of an autoimmune disease or an inflammatory-related disease, comprising:

administering a therapeutically effective amount of a pharmaceutical composition comprising CD83⁺ mesenchymal stem cells/CD83⁺ overexpressed mesenchymal stem cells to a subject suffering from the autoimmune disease or the inflammatory-related disease. In some embodiments, the pharmaceutical composition further comprises CD83⁺PD-L1⁺ mesenchymal stem cells.

As used herein, “pharmaceutical composition” refers to drugs used for the prevention or treatment of disease. When used for the prevention or treatment of disease, “pharmaceutical composition” usually refers to a unit dose form and may be prepared by any of the manners well known in the pharmaceutical field. All manners include steps to combine the active ingredient with excipients constituting one or more accessory ingredients.

Embodiments of the present disclosure provide immunosuppressive or anti-inflammatory function-prone CD83⁺ mesenchymal stem cells (MSCs), and immunosuppressive or anti-inflammatory function enhanced CD83⁺PD-L1⁺ MSCs and preparation methods thereof. Embodiments of the present disclosure also provide use of the immunosuppressive or anti-inflammatory function-prone CD83⁺ MSCs and the immunosuppressive or anti-inflammatory function enhanced CD83⁺PD-L1⁺ MSCs.

Embodiments of the present disclosure further provide use of CD83⁺ MSCs in the preparation of drugs for preventing/treating of at least one of autoimmune disease or inflammatory-related disease.

Based on the heterogeneity and functional plasticity of the MSCs, anti-inflammatory and pro-inflammatory function-prone subpopulations may exist in the MSCs, and the balance of the anti-inflammatory and pro-inflammatory subpopulations correlates with regression and treatment resistance in inflammatory/immunity-related diseases. Therefore, it is important to clarify the information on the cell surface markers and functional characteristics of the anti-inflammatory and pro-inflammatory function-prone subpopulations of the MSCs, and then to obtain the high-purity anti-inflammatory function-prone subpopulations of the MSCs or anti-inflammatory specialized MSCs without pro-inflammatory function-prone subpopulations by the sorting techniques such as magnetic beads or flow assays, and to develop new MSCs drugs for autoimmune/inflammation-related diseases by using anti-inflammatory function-prone subpopulations of MSCs or anti-inflammatory specialized MSCs as seed cells, which greatly improves the clinical efficacy of the MSCs and has a high druggability. To this end, the present disclosure identifies a subpopulation of CD83-positive MSCs by single-cell sequencing, and the subpopulation of cells have more sensitive anti-inflammatory enhancement effect on immunosuppressive or anti-inflammatory function mediated by immune factors, which is defined as an “immunosuppressive and anti-inflammatory function-prone CD83⁺-MSCs subpopulation”. Therefore, the development of new MSCs drugs for autoimmune/inflammatory diseases based on the subpopulation of MSCs greatly improves the clinical efficacy of MSCs and has a high druggability.

Embodiments of the present disclosure also provide use of CD83⁺ overexpressed mesenchymal stem cells in the preparation of drugs for preventing/treating of at least one of autoimmune disease or inflammatory related disease.

In some embodiments, CD83⁺ MSCs/CD83⁺ overexpressed MSCs are capable of increasing the secretion of at least one of an anti-inflammatory factor or an immunosuppressive factor.

In some embodiments, the immunosuppressive factor includes at least one of IL-10, IDO, PD-L1, GDNF, IL-10Rα, IL-2Rγ, IL-4, IL-4R, IL-17B, LIF, GM-CSFRα, TIMP-3, and IL-2Rβ.

In some embodiments, the anti-inflammatory factor includes at least one of IL-10, IDO, PD-L1, GDNF, IL-10Rα, IL-2Rγ, IL-4, IL-4R, IL-17B, LIF, GM-CSFRα, TIMP-3, and IL-2Rβ.

In some embodiments, the autoimmune disease includes at least one of rheumatoid arthritis, scleroderma, systemic lupus erythematosus, ankylosing spondylitis, myasthenia gravis, ulcerative colitis, or the like; and the inflammatory-related disease includes at least one of sepsis, degenerative arthritis, neonatal bronchopulmonary dysplasia pneumonia, or the like.

Embodiments of the present disclosure also provide a preparation method for CD83⁺ MSCs/CD83⁺ overexpressed MSCs, comprising following steps.
(a) isolating and culturing the MSCs;
(b) sorting and obtaining immunosuppressive or anti-inflammatory function-prone natural CD83⁺ MSCs/CD83⁺ overexpressed MSCs by magnetic beads coupled with anti-CD83 antibody and a flow sorting technology, or a lentiviral gene transduction technology.

In some embodiments, the magnetic bead and the flow sorting technology refer to sorting and obtaining the natural CD83⁺ MSCs by the magnetic bead coupled with the anti-CD83 antibody and the flow sorting technology, and a sorting reagent includes the magnetic bead coupled with the anti-CD83 antibody or a CD83 flow sorting antibody.

In some embodiments, the lentiviral gene transduction technology refers to obtaining artificial CD83⁺ overexpressed MSCs modified with human CD83 gene by the lentiviral gene transduction technology, and the human CD83 gene transduction technology includes, but is not limited to, lentiviral, adenoviral, traditional plasmid expression vectors, and other gene modification transduction technologies.

In some embodiments, the MSCs are derived from human adipose, dental pulp, bone marrow, umbilical cord, placenta, or cord blood.

In some embodiments, isolating and culturing the MSCs in step (a) includes: collecting sample tissues for washing; separating the tissues; placing the separated tissues in saline for washing, and cutting and weighing, transferring them into a centrifuge tube for centrifugation, and discarding the supernatant; adding MSCs medium to the centrifuge tube, and culturing them for a certain period of time; and when the primary cells grow to a cell density greater than 80%, digesting and dissociating the primary cells and performing a secondary culture to obtain a mixed MSC population containing CD83⁺ MSCs.

Embodiments of the present disclosure also provide use of CD83⁺PD-L1⁺ mesenchymal stem cells in the preparation of drugs for preventing/treating at least one of autoimmune disease or inflammatory related disease.

In some embodiments, CD83⁺PD-L1⁺ MSCs are capable of increasing the secretion of at least one of anti-inflammatory or immunosuppressive factors.

In some embodiments, the immunosuppressive factor includes at least one of IL-10, IDO, PD-L1, GDNF, IL-10Rα, IL-2Rγ, IL-4, IL-4R, IL-17B, LIF, GM-CSFRα, TIMP-3, and IL-2Rβ.

In some embodiments, the anti-inflammatory factor includes at least one of IL-10, IDO, PD-L1, GDNF, IL-10Rα, IL-2Rγ, IL-4, IL-4R, IL-17B, LIF, GM-CSFRα, TIMP-3, and IL-2Rβ.

In some embodiments, the autoimmune disease includes at least one of rheumatoid arthritis, scleroderma, systemic lupus erythematosus, ankylosing spondylitis, myasthenia gravis, ulcerative colitis, or the like; and the inflammatory-related disease includes at least one of sepsis, degenerative arthritis, neonatal bronchopulmonary dysplasia pneumonia, or the like.

Embodiments of the present disclosure also provide a preparation method for CD83⁺PD-L1⁺ MSCs, comprising obtaining immunosuppressive or anti-inflammatory function enhanced CD83⁺PD-L1⁺ MSCs by inducing and culturing CD83⁺ mesenchymal stem cells using an immune factor inducing complex.

In some embodiments, the immune factor inducing complex includes an IFN-γ immune factor and a TNF-α immune factor, a concentration of the IFN-γ immune factor is within a range of 5-20 ng/ml, and a concentration of the TNF-α immune factor is within a range of 0-20 ng/mL.

In some embodiments, the immune factor inducing complex includes the IFN-γ immune factor and the TNF-α immune factor, the concentration of the IFN-γ immune factor is 20 ng/ml, and the concentration of the TNF-α immune factor is within a range of 5-20 ng/mL.

In some embodiments, the immune factor inducing complex further includes an albumin excipient.

In some embodiments, a timing of the addition of the immune factor inducing complex is 3-5 generations of CD83⁺ mesenchymal stem cells proliferation culture to 70-80% of cell fusion degree.

In some embodiments, the induced culture includes: inducing the CD83⁺ MSCs for a period of 18-24 h by the immune factor inducing complex, and then withdrawing the inducing complex and continuing culturing the induced CD83⁺ MSCs for at least 6 h.

Embodiments of the present disclosure also provide a quality assessment method for the CD83⁺PD-L1⁺ MSCs obtained by the preparation method, and the positive expression rates of CD83 and PD-L1 receptors in the CD83⁺PD-L1⁺ MSCs are used as quality control indicators of the CD83⁺PD-L1⁺ mesenchymal stem cells.

In some embodiments, the positive expression rate of the CD83 is at least greater than or equal to 70%, and the positive expression rate of the PD-L1 receptor is greater than or equal to 30%.

Embodiments of the present disclosure also provide a kit. The kit is effectively used for evaluating a quality requirement of the CD83⁺PD-L1⁺ MSCs by detecting the positive expression rates of the CD83 and the PD-L1 receptors with the quality assessment method; and the kit further comprises the immune factor inducing complex.

In some embodiments of the present disclosure, the CD83⁺ MSCs are first identified and validated, and the CD83⁺ MSCs are more sensitive to the expression regulation of IFN-γ-mediated anti-inflammatory factor, manifested by higher expression of IL-10, GDNF, IL-10Rα, IL-2Rγ, IL-4, IL-4R, IL-17B, LIF, GM-CSFRα, TIMP-3, IL-2Rβ and other anti-inflammatory factors after stimulation with the IFN-γ. Therefore, it indicates that the CD83⁺-MSCs subpopulations are more sensitive to the IFN-γ-mediated anti-inflammatory function-prone of MSCs and is considered as an anti-inflammatory function-prone subpopulation. In addition, the CD83⁺ MSCs are induced by the IFN-γ alone and/or combined with TNF-α to obtain immunosuppressive or anti-inflammatory enhanced CD83⁺PD-L1⁺ MSCs, and the CD83⁺PD-L1⁺ MSCs can highly express immunosuppressive or anti-inflammatory factors such as PD-L1, IDO, IL-10, GDNF, IL-10Rα, IL-2Rγ, IL-4, IL-4R, IL-17B, LIF, GM-CSFRα, TIMP-3, IL-2Rβ, etc., which has a strong immunosuppressive or anti-inflammatory function. On the basis of the present disclosure, immunosuppressive or anti-inflammatory function-prone CD83⁺-MSCs or immunosuppressive or anti-inflammatory function enhanced CD83⁺PD-L1⁺-MSCs obtained by induction are used in the treatment of inflammatory or immune-related diseases. These two MSCs can highly express the above anti-inflammatory factors and/or immunosuppressive factors, while eliminating the interference of pro-inflammatory MSCs subpopulations, so CD83⁺ MSCs/CD83⁺PD-L1⁺ MSCs have better immunosuppressive and anti-inflammatory effects on the treatment of immune and inflammatory related diseases so as to have better clinical efficacy in the treatment of inflammatory or immune-related diseases. Meanwhile, using a mice model of rheumatoid arthritis and a mice model of CLP sepsis, the effectiveness and safety of the immunosuppressive or anti-inflammatory enhanced CD83⁺PD-L1⁺-MSCs/immunosuppressive or anti-inflammatory function-prone CD83⁺ MSCs/CD83⁺ overexpressed MSCs in the treatment of immune or inflammation-related diseases are evaluated before clinical practice, and they are proved to be safe and effective.

In some embodiments of the present disclosure, the CD83⁺ MSCs induced by the IFN-γ alone can up-regulate the expression of immunosuppressive factor IDO and immune checkpoint receptor PD-L1 in CD83⁺-MSCs at the transcriptional level, significantly enhancing the anti-inflammatory function-prone effect of the IFN-γ-mediated MSCs. The CD83⁺ MSCs induced by the IFN-γ combined with TNF-α can significantly up-regulate the protein expression of the IDO and the PD-L1 at both the transcriptional level and translation level, enhancing the immunomodulatory and anti-inflammatory functions of the MSCs. The present disclosure innovatively proposes that CD83⁺-MSCs are induced using the TNF-γ combined with the IFN-α, which can significantly up-regulate the expression of the immunosuppressive factor IDO and the immune checkpoint receptor PD-L1 at the transcriptional and translational levels, to enhance the immunomodulatory and anti-inflammatory ability of MSCs from different perspectives and to enhance their therapeutic efficacy in immune and inflammation-related diseases.

In some embodiments of the present disclosure, immunosuppressive or anti-inflammatory function-prone natural or artificial CD83⁺ overexpressed MSCs are also successfully sorted or prepared by using magnetic beads coupled with anti-CD83 antibody and the flow sorting technique, or the lentiviral gene transduction technique. Meanwhile, on this basis, the immunosuppressive or anti-inflammatory function enhanced CD83⁺PD-L1⁺ MSCs are successfully obtained by inducing culture of the CD83⁺ MSCs by using the immune factor inducing complex.

In some embodiments of the present disclosure, immunosuppressive or anti-inflammatory function enhanced CD83⁺PD-L1⁺-MSCs are innovatively used to treat sepsis, which can significantly reduce the mortality rate and inflammatory response of the sepsis; and immunosuppressive or anti-inflammatory function enhanced CD83⁺PD-L1⁺-MSCs are used to treat collagen induced arthritis, which can also significantly improve the clinical symptoms of arthritis in mice. At the same time, according to the induction process of the anti-inflammatory CD83⁺PD-L1⁺-MSCs, an anti-inflammatory CD83⁺PD-L1⁺-MSCs induction kit is further developed, which can be readily promoted and applied.

In view of the role of PD-L1 signal in the anti-inflammatory function of MSCs and the fact that the PD-L1 signal is a membrane receptor extremely easy to be rapidly detected by flow cytometry, the PD-L1 expression can be used as quality control indicators for the assessment of anti-inflammatory function of MSCs, which provides technical support for the further development of anti-inflammatory CD83⁺PD-L1⁺-MSCs induction kits that can be easily promoted and applied.

The technical solutions of the present disclosure are described in further detail below in connection with specific embodiments. It should be understood that the following embodiments are only exemplary for illustrating and explaining the present disclosure and should not be construed as a limitation on the scope of protection of the present disclosure. Any technology realized based on the foregoing contents of this disclosure is covered by the scope of protection intended by this disclosure.

The experimental methods used in the following embodiments are conventional, if not otherwise specified. Materials, reagents, etc. used in the following embodiments are commercially available if not otherwise specified.

### EMBODIMENTS

### Embodiment 1 Isolation, Culture, and Identification of Human Umbilical Cord MSCs

The present embodiment provides a preparation process for the human umbilical cord MSCs, including: isolation, culture, and identification of the human umbilical cord MSCs from neonatal umbilical cord, including the following steps. 1. Isolation and culture of MSCs

Isolation and culture of the human umbilical cord MSCs (hUC-MSCs): fresh umbilical cord samples were collected under aseptic conditions (from healthy mothers who were negative for hepatitis B, hepatitis C, HIV, and syphilis and had no hereditary diseases, and who had passed the application for ethical use and obtained informed consent for the collection of the samples), and the samples were washed with saline in an ultra-clean bench in a GMP laboratory, to wash away the blood residues and other substances from the surface of the samples, and then the washed samples were transferred to an alcohol dish for about 1 min, and then transferred to a clean saline dish to wash the alcohol from the umbilical cord. The two ends of the umbilical cord were cut to exclude the coagulated blood within the umbilical cord, and then the umbilical cord was cut into umbilical cord blocks with a length of 2 cm. The small umbilical cord blocks were placed in clean saline dishes for washing again, to further cleanse the coagulated blood of the umbilical cord. A clean petri dish was taken and used for separating Wharton's Jelly. The blood vessels (one vein and two arteries) were removed from each umbilical cord block in turn with hemostatic forceps, and then the tissue skin and the tissue block were separated from the remaining umbilical cord tissue along the edge (the tissue block was the Wharton's Jelly). Then the separated colloid was placed in a clean saline dish; after separating the colloid, the large strips of Wharton's Jelly was separated as finely as possible using forceps, and finally the colloid was washed again in a clean saline dish and transferred into a sterilized sterile vial. The Wharton's Jelly was cut as much as possible with scissors and weighed, the cut Wharton's Jelly was divided into 15 mL centrifuge tubes according to the weight, with 0.5 g per tube, 10 mL of saline was added per tube, the Wharton's Jelly solution was centrifuged at 2000 rpm for 5 min, and the supernatant was discarded afterward. Two 25 cm² culture bottles were taken and labeled with the name of the primary generation of the culture, the date of the culture, and a number or a bar code. 4 mL of serum-free Ultra CULTURE™ medium (containing 2 mM L-glutamine) was added to each of the two centrifuge tubes (4 mL of medium for every 0.5 g of tissue block), covered, and shaken well. The culture bottles were placed in an incubator (incubator at 37° C. and 5% CO₂) for incubating for 5-7 days, and then fresh medium was added. When the primary cells grew to a cell density of greater than or equal to 80%, the adherent cells were digested by TrypLE, after centrifugation and liquid exchange, a secondary culture was performed. The hUC-MSCs used in the present embodiment were the 3-5th generation cells, which were sourced from the original seed bank cells.

### 2. Identification of the MSCs

### (1) Identification of Surface Marker Molecules of the MSCs

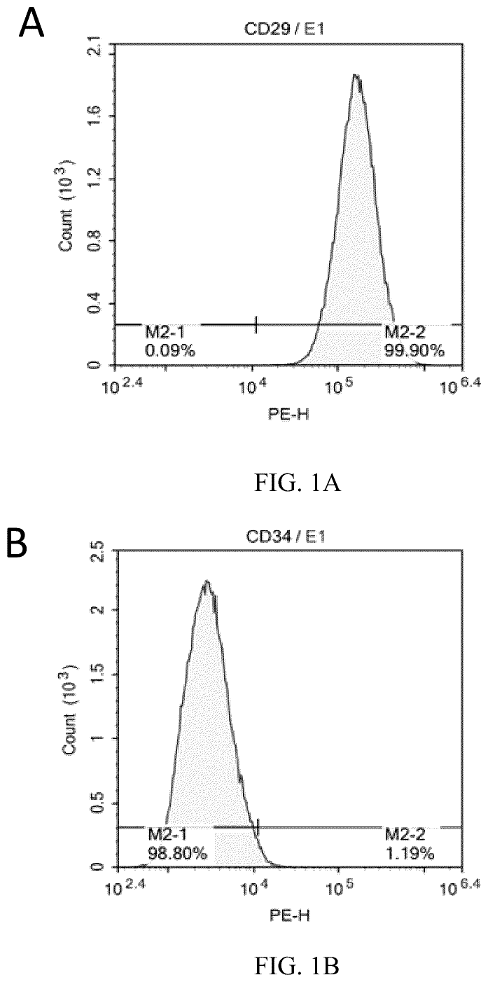

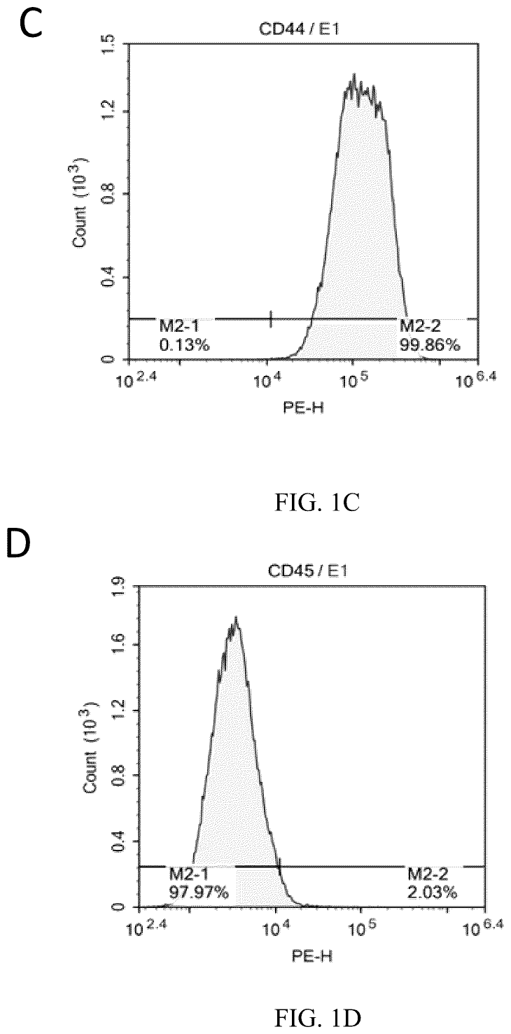

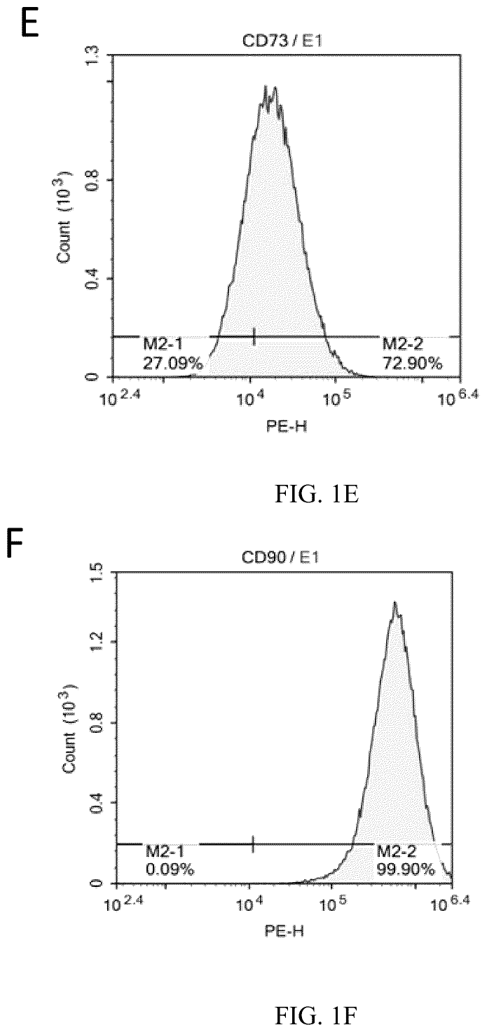

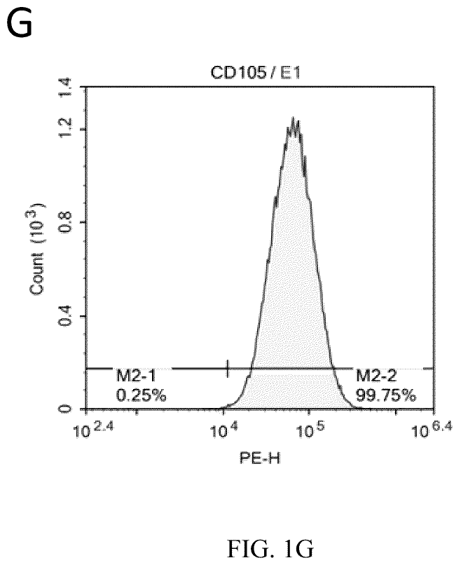

Expression levels of the surface marker molecules were detected in isolated and cultured MSCs by a flow cytometry. Stem cell marker molecules detected by the flow cytometry included the positive molecules CD73, CD44, CD90, and CD105, and the negative molecules CD29, CD34, and CD45. The identification results show that the expression of the surface marker molecules of hUC-MSCs obtained by in vitro purification and culture is positive for CD44, CD73, CD90, and CD105, and negative for CD29, CD34, and CD45, which meets the criteria for the definition of MSC of the International Society for Cellular Therapy. The flow cytometry results of surface marker molecules with negative/positive expression are shown in FIGS. 1A-1G.

(2) Identification of triple lineage differentiation ability of the MSCs

Osteogenesis induction differentiation: the 3rd generation of wild-type hUC-MSCs were taken and inoculated in six-well plates after digesting and counting, and a certain amount of complete medium was added into each well, and then when the cell density reached 60%-70%, the medium was replaced with human umbilical cord mesenchymal stem cell osteogenic induced differentiation complete medium from OriCell, and the medium was replaced with a fresh osteogenic induced differentiation complete medium at regular intervals according to the cell growth. After about 2-4 weeks of culture, 1 mL of alizarin red staining solution was added to each well, and the staining effect of osteogenic staining was observed under the microscope after washing with PBS twice.

Chondrogenesis induction differentiation: the 3rd generation of wild-type hUC-MSCs were taken, after digestion, 4×10⁵ cells were transferred to a centrifuge tube for centrifugation and the supernatant was discarded; 0.5 mL of kit premix was added (the kit premix was commercially purchased, and in the present embodiment, the kit premix was one of the components of the Human Umbilical Cord Mesenchymal Stem Cells Chondrogenic Induced Differentiation Kit from OriCell, a model of HUXUC-90041), the hUC-MSCs precipitate was resuspended and centrifuged again at room temperature, and the supernatant was discarded. After that, the cells were re-suspended with 0.5 mL human umbilical cord mesenchymal stem cells chondrogenic induced differentiation complete medium from OriCell, and the cells were inoculated into six-well plates, and then cultured in an incubator. The medium was replaced with fresh chondrogenic induced differentiation complete medium at regular intervals. After 28 days of continuous induction culture, the chondrocyte spheres were subjected to fixation with formalin and paraffin section, and Alisin blue staining was applied to the sections, and the excess dye was washed away by PBS, and the staining effect of the chondrogenic was assessed by observation under the microscope.

Lipogenesis induced differentiation: the 3rd generation of wild-type hUC-MSCs were taken, digested, and counted, and then inoculated into six-well plates, a certain amount of complete medium was added to each well, and the plates were placed in an incubator to culture until the cell density reached 100%. The medium in each well was replaced with liquid A of human umbilical cord mesenchymal stem cells adipogenic induced differentiation medium from OriCell; after 72 h of induction, the liquid A was aspirated from the six-well plates and the liquid B was added; after 24 h, the liquid B was aspirated and then the liquid A was added for culture. After that, the cells were cultured in the liquid A and liquid B alternately for 3-5 cycles (about 12-20 days), and then the culture was maintained in the liquid B for 7 days (changing the liquid for once every 3 days). After that, the cells were fixed with paraformaldehyde for 30 min, and then 1 mL of oil-red O dye was added to each well for staining after washing with PBS, and the staining effect of adipogenic was evaluated by microscopic observation.

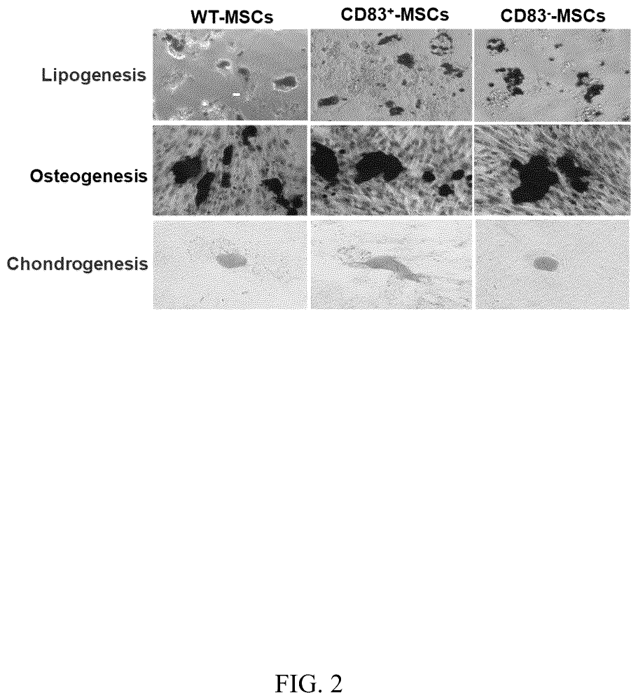

The identification results of the triple lineage differentiation ability of wild-type mesenchymal stem cells (WT-MSCs) are shown in FIG. 2, from which it can be seen that the wild-type hUC-MSCs (labeled as WT-MSCs in the figure) obtained in the present embodiment have the ability to differentiate into the triple lineages of osteoblasts, chondrocytes, and adipocytes under the specific induced culture conditions, which indicates that wild-type hUC-MSCs obtained by culture and purification in the present embodiment meets the standards of the International Society for Cellular Therapy for human MSCs.

### Embodiment 2 Sorting, Preparation, Culture, and Identification of CD83⁺-MSCs

The present embodiment provides a preparation process for a subpopulation of anti-inflammatory function-prone CD83⁺ human umbilical cord MSCs.

Anti-inflammatory function-prone CD83⁺ human umbilical cord MSCs were obtained from a seed bank of wild-type human umbilical cord MSCs by magnetic beads or flow sorting or gene-modified overexpression, including the following steps. 1. Sorting and preparation of CD83⁺ MSCs

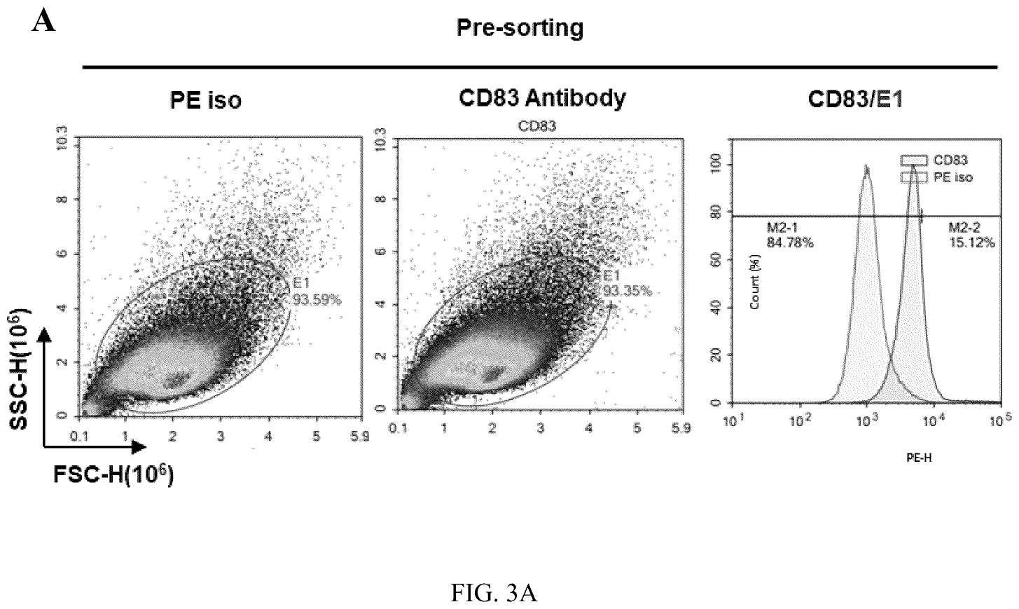

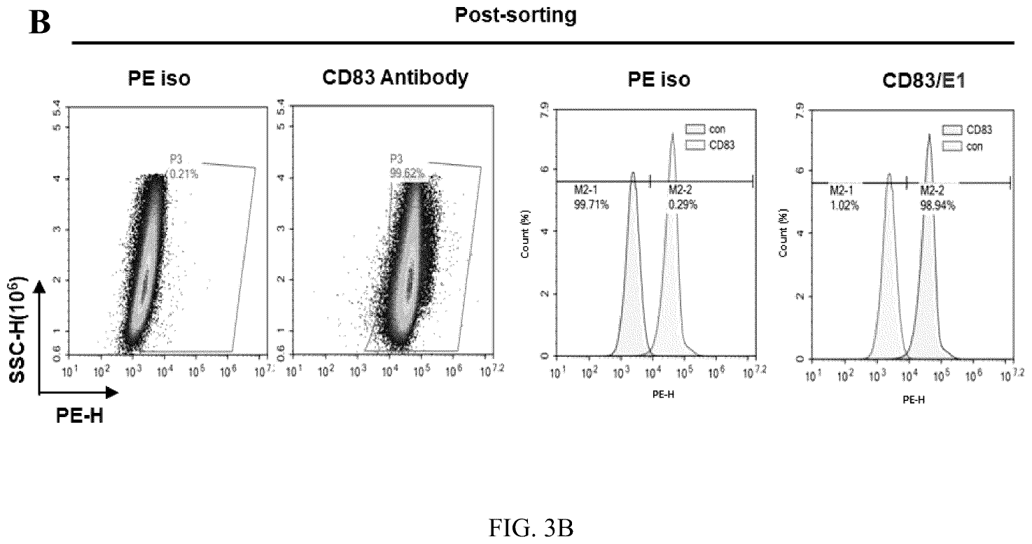

(1) Sorting of CD83⁺ MSCs by magnetic beads: on the basis of the embodiment 1, LS sorting column combined with magnetic sorter was used. The hUC-MSCs were cultured to a fusion degree of 80% and digested using 0.25% trypsin to obtain the hUC-MSCs suspension. The hUC-MSCs suspension was washed by PBS for 1-2 times and centrifuged, and the PBS was discarded. The hUC-MSCs were incubated with the anti-CD83 monoclonal antibody in the dark for 15 min, and the non-specific binding anti-CD83 antibody were washed away, then the magnetic beads were added and continued to incubate in the dark for 10 min, after incubation, the sample passed through the column, and the cells labeled with the magnetic beads were adsorbed on the sorting column under the action of the magnetic field, and the negative cells not bound to the anti-CD83 antibody flowed to the test tubes, i.e., CD83⁻-MSCs. The LS sorting column was taken off from the magnetic rack and rinsed again, the hUC-MSCs bound with magnetic beads and anti-CD83 antibody were eluted because of the absence of magnetic force, thus obtaining CD83-positive MSCs, i.e., CD83⁺-MSCs. The CD83⁺-MSCs were collected by centrifugation, and a portion of them was identified by flow cytometry to determine a positive expression rate of CD83 on the membrane surface of the isolated CD83⁺-MSCs, which was used as an indicator of the purity of cell sorting of CD83⁺-MSCs, and the rest of the cells were cultured for another generation, and then the purity was identified according to flow cytometry analysis, and the purity was greater than or equal to 80%, and the cells were frozen and stored as a CD83⁺-MSCs cell seed bank. FIGS. 3A-3B show the positive expression rate of CD83 on the membrane surface of hUC-MSCs before (FIG. 3A) and after (FIG. 3B) sorting using magnetic bead. The positive expression rate is about 15% before sorting, and the purity was more than 98% after sorting, suggesting that the magnetic bead sorting could obtain high purity CD83⁺-MSCs.

(2) Flow sorting of CD83⁺ MSCs: in addition to the application of magnetic bead sorting to obtain high-purity CD83⁺-MSCs, other sorting techniques such as flow sorting was also used to obtain CD83⁺-MSCs, the sorting scheme was as follows. FITC or PE fluorescent-labeled anti-CD83 antibody was incubated with the hUC-MSCs at room temperature for 30 min, and the cells were resuspended into single-cell suspensions, and then the flow cell sorting parameters were set according to the operating requirements of the flow sorter, the cell were sampled for sorting to obtain the high-purity CD83⁺-MSCs, and after one generation of cell culture, flow analysis was performed to identify the purity, the purity was greater than or equal to 80%, and the cells were frozen and stored as a CD83⁺-MSCs cell seed bank.

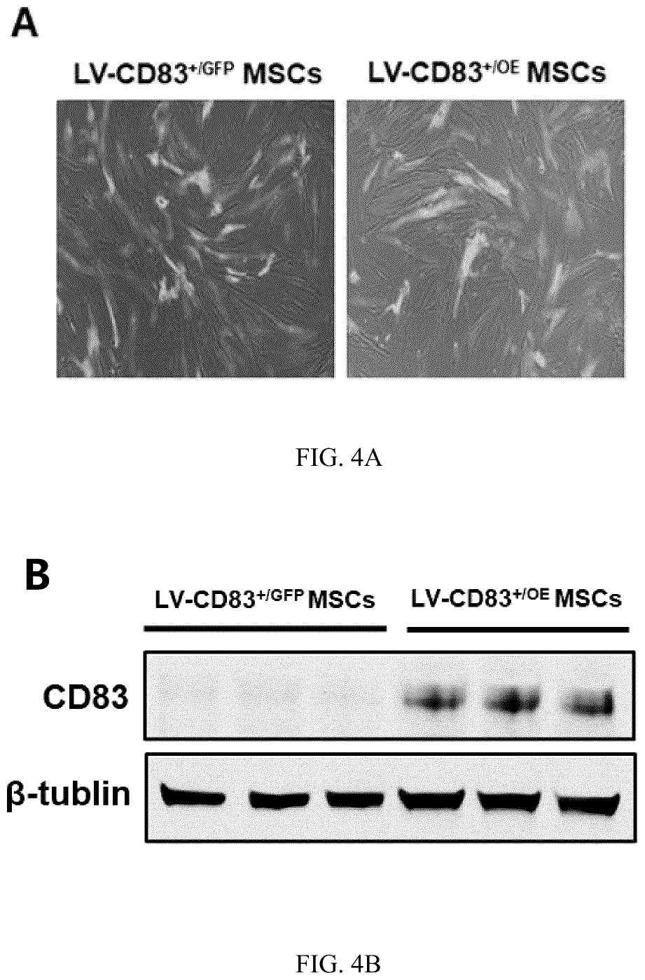

(3) Preparation of artificial CD83 overexpressed MSCs by human CD83 gene modification: LV-CD83 lentivirus was used to construct stable CD83 overexpressed MSCs, and the sequence of the viral vector components was as follows: Ubi-MCS-3FLAG-CBh-gcGFP-IRES-puromycin, and the sequence of CD83 was found in the GenBank (gene number: NM_004233.4), and LV-CD83 lentiviral expression vector was constructed, and then the virus was packaged and purified for use. MSCs were prepared into cell suspension with complete medium and inoculated into T25 culture bottoms prior to transfection, and incubated at 37° C., 5% CO₂ for 16-24 h until the cell fusion degree was about 40%, and lentiviral transfection was performed. HiTransG A or HiTransG P infection solution were selected, the appropriate amount of virus was added according to the cellular MOI value (the MOI value of 10) and viral titer, and then placed in the incubator at 37° C. and 5% CO₂ to culture for 16 h, and then the medium was replaced with complete medium for further incubation. After about 72 h of infection, the efficiency of cellular viral infection was observed under the fluorescence microscope, and due to lentivirus-infected cells with puromycin resistance, 1 mg/mL puromycin was added to kill the uninfected cells, purification was performed to screen CD83 overexpressed cell lines, and the cells were passaged and frozen for backup after screening was completed. FIG. 4A shows a result of fluorescence microscopy of CD83 overexpression with GFP fluorescence, and FIG. 4B shows the result of CD83 protein expression in MSCs detected by immunoblotting with anti-Flag tag antibody.

### 2. Identification of CD83⁺ MSCs

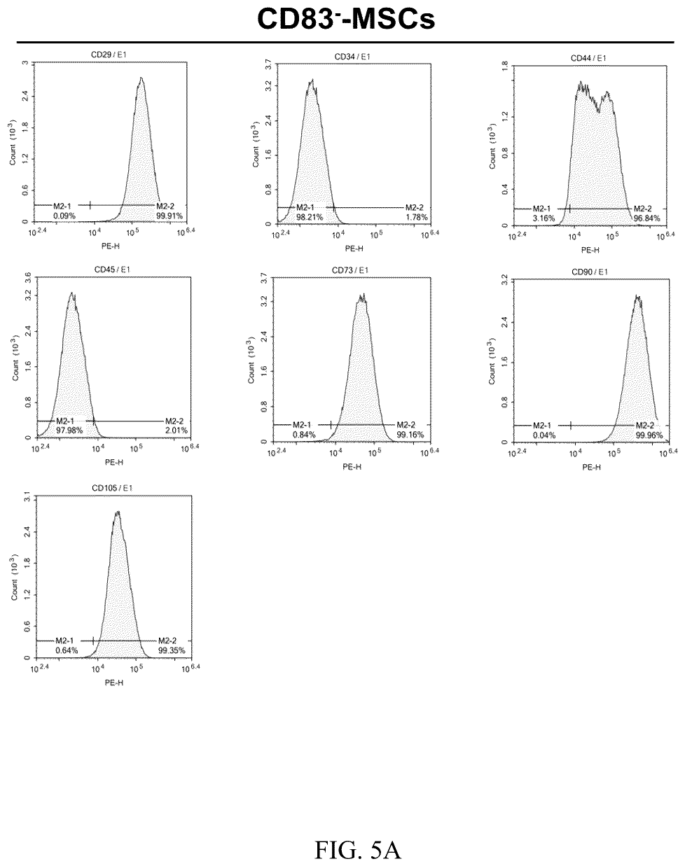

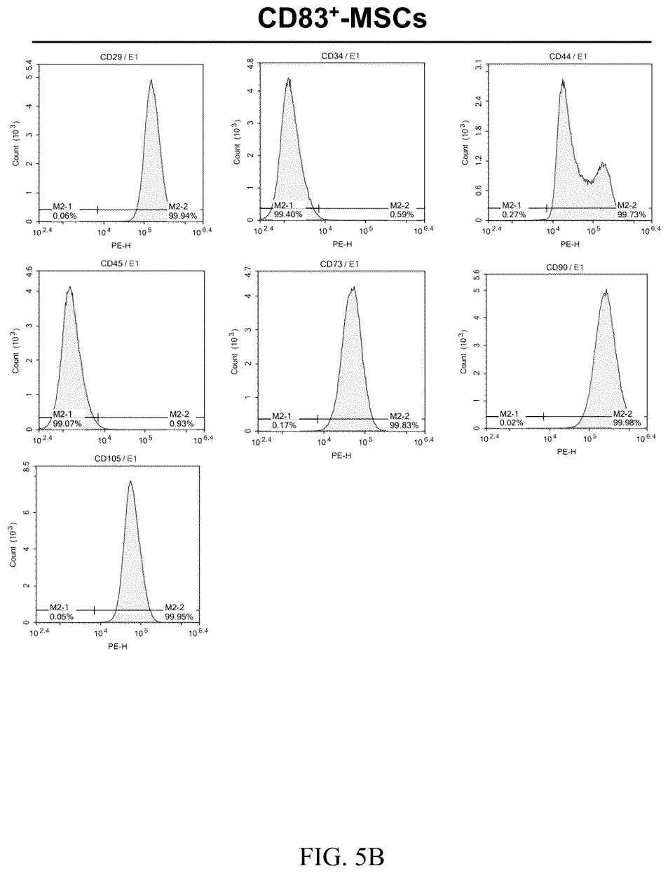

(1) Identification of surface marker molecules for CD83⁺ MSCs: the experimental steps referred to the section 2-(1) in Embodiment 1, and the difference was that MSCs were replaced with CD83⁺ or CD83-MSCs. The flow results show that there is no difference in the expression of surface marker molecules between the CD83⁻-MSCs or CD83⁺-MSCs of the present embodiment and the wild-type hUC-MSCs, with the expression of CD44, CD73, CD90, and CD105 being positive and CD29, CD34, and CD45 were negative, meeting the criteria for MSCs as defined by the International Society for Cellular Therapy. The flow assay results of surface marker molecules with negative/positive expression are shown in FIG. 5.

(2) Identification of triple lineage differentiation ability of CD83⁺ MSC: the experimental steps referred to the section 2-(2) in Embodiment 1, and the difference was that wild-type MSCs were replaced with CD83⁻-MSCs or CD83⁺-MSCs. The results of the trilineage differentiation experiments are shown in FIG. 2. It can be seen form FIG. 2 that the trilineage differentiation ability of the CD83⁻-MSCs or CD83⁺-MSCs obtained in the present embodiment is consistent with wild-type MSCs hUC-MSCs (labeled WT-MSCs in the figure), and all of them have the ability of trilineage differentiation to osteoblasts, chondrocytes, and adipocytes under specific induced culture conditions, indicating that the CD83⁺-MSCs also meet the criteria for MSCs as defined by the International Society for Cellular Therapy.

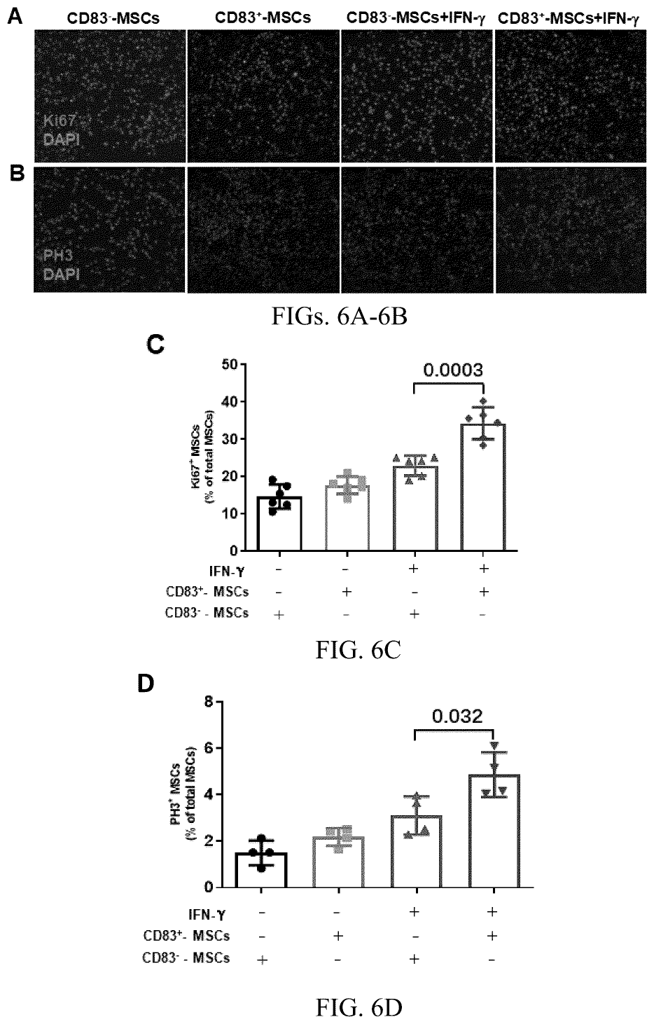

(3) Identification of the proliferative function of CD83⁺ MSCs: based on the principle that Ki-67 protein is a cell cycle-related nuclear protein, which mainly expresses in the proliferative and divisive phases of cells, but not expressed in resting cells (GO phase), the detection of the content of Ki-67 can reflect the proliferative activity of cells. In addition, phosphorylated histone H3 (PH3) is highly expressed during cell mitosis and can be used as a stable indicator of cell proliferation. CD83⁻-MSCs and CD83⁺-MSCs were taken in logarithmic growth phase and prepared into single cell suspension after digestion using 0.25% trypsin. Aseptically cleaned coverslips were added to 6-well plates, and then CD83⁻-MSCs and CD83⁺-MSCs cells were inoculated to 6-well plates at 1×10⁵ cells/well for incubation for 24 h, when the cell fusion degree reached to 60-70%, the coverslips crawling with cells were taken out, washed twice with PBS, and fixed by adding 4% paraformaldehyde for 1 h-2 h. The fixed cell slides were covered with PBS for 15 min, then washed three times with PBS; cell permeabilization solution (0.1% Triton-X) was added on the slides, and the slides was left on ice for 5 min; the slides were soaked in washing solution for 5 min and washed three times, then hydrogen peroxide working solution was added dropwise to cover the samples, and the slides were left on ice for 5-10 min; the slides were soaked in PBS for 2 min and washed three times, and 50 μL of ready-to-use goat serum was added dropwise to each slides, and incubated in a wet box at 37° C. for 10 min, then the slides were soaked in washing solution for 2 min and washed for 3 times; 100 UL of primary antibody against Ki67 and PH3 (a dilution ratio of Ki-67 and PH3 antibody of 1:100) was added and incubated at 37° C. for 1 h; the slides were soaked in PBS for 2 min and washed for 3 times, and then 50 μL of ready-to-use HRP-labeled secondary antibody IgG was added dropwise to cover the samples. After incubation for 10 min at 37° C. in a wet box, the slides were soaked in PBS for 2 min and washed for 5 times, then one drop of prepared DAB coloring solution was added to each slice, and the color was developed at room temperature for 2-5 min; the slides were soaked in PBS for 2 min and washed for 5 times, then the staining depth was observed under the microscope, and the staining was discontinued immediately when the staining was completed, the slides were gently rinsed for 15 min with the tap water and the coloring reaction was terminated with distilled water; the cells of slides were stained for DNA for 3 min with DAPI at a final concentration of 100 ng/mL, soaked in PBS for 2 min, washed 5 times, sealed, and then examined under a light microscope and photographed. FIG. 6A shows the Ki67 staining result of cell slides, FIG. 6B shows the PH3 staining result of cell slides, and FIG. 6C shows the result of Ki67 and PH3 positive statistic of FIGS. 6A-6B. As shown in FIGS. 6A-6C, under the condition without IFN-γ stimulation, the proliferative ability between the two groups of CD83⁻-MSCs and CD83⁺-MSCs is weaker and not significantly different, but after stimulation by the IFN-γ, the proliferative ability of CD83⁺-MSCs is significantly enhanced compared with CD83⁻-MSCs.

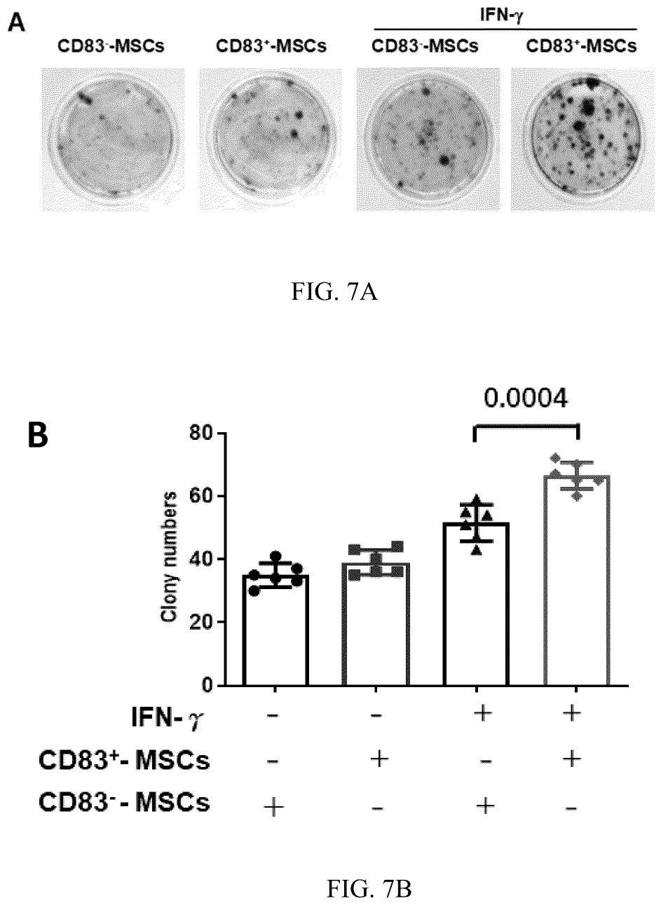

(4) Identification of clone formation ability of CD83⁺ MSCs: CD83⁻-MSCs and CD83⁺-MSCs cells in logarithmic growth phase were taken and prepared into single cell suspension after digestion with 0.25% trypsin. Approximately 200 CD83⁻-MSCs and CD83⁺-MSCs were inoculated in a T25 culture dish, and the cells were blown or manually rotated to make a uniform dispersion, and then placed in a cell culture incubator for static culture for 2-3 weeks. After the appearance of spheres distinguishable by naked eye in the culture dish, the supernatant was discarded, washed with PBS, fixed for 10 min, stained with crystal violet for 30 min, washed with PBS, and photographed for observation. FIGS. 7A-7B show the clonal proliferative ability of MSCs with or without IFN-γ stimulation. As shown in FIGS. 7A-7B, under the condition without IFN-γ stimulation, the CD83⁻-MSCs and CD83⁺-MSCs have no significant difference in clonal proliferation ability, but after the IFN-γ stimulation, the clonal proliferation ability of CD83⁺-MSCs is significantly enhanced compared with that of CD83⁻-MSCs.

### (5) Identification of CD83⁺ MSC Migration Function

(a) Cell scratching experiment: use 0.25% trypsin to digest CD83⁻-MSCs and CD83⁺-MSCs cells in logarithmic growth phase to make a single-cell suspension of 5×10⁵ cells/mL, inoculate 2 mL of cell suspension per well in a 6-well plate, put it in a cell incubator and incubate it for 24 h. When the fusion rate was 100%, draw a straight line with the tip of the 100 μL lance (aided by a ruler). PBS was used to wash away the scratched cells, and the IFN-γ was added after replacing the new medium, and then the cells were placed in the cell culture incubator for further incubation, and then pictures were taken at 0 h, 6 h, 12 h, and 24 h after scratching, respectively, and the image area of scratches was calculated using Image J software.

(b) Transwell experiment: digest CD83⁻-MSCs and CD83⁺-MSCs cells in logarithmic growth phase with 0.25% trypsin to prepare a single-cell suspension of 1×10⁵ cells/mL, place the suspension in small wells of a Transwell, with 500 μL of suspension in each well, place the Transwell in a 24-well plate, add 1 mL of culture medium and stimulating factor IFN-γ to each well in a 24-well plate, place it in a cell incubator for 24 h. Discard the culture medium, fix the cells in cell fixative for 10 min, wash twice with PBS, stain with crystal violet for 30 min, and wash twice with PBS, then put the cells under the microscope to observe the cell migration.

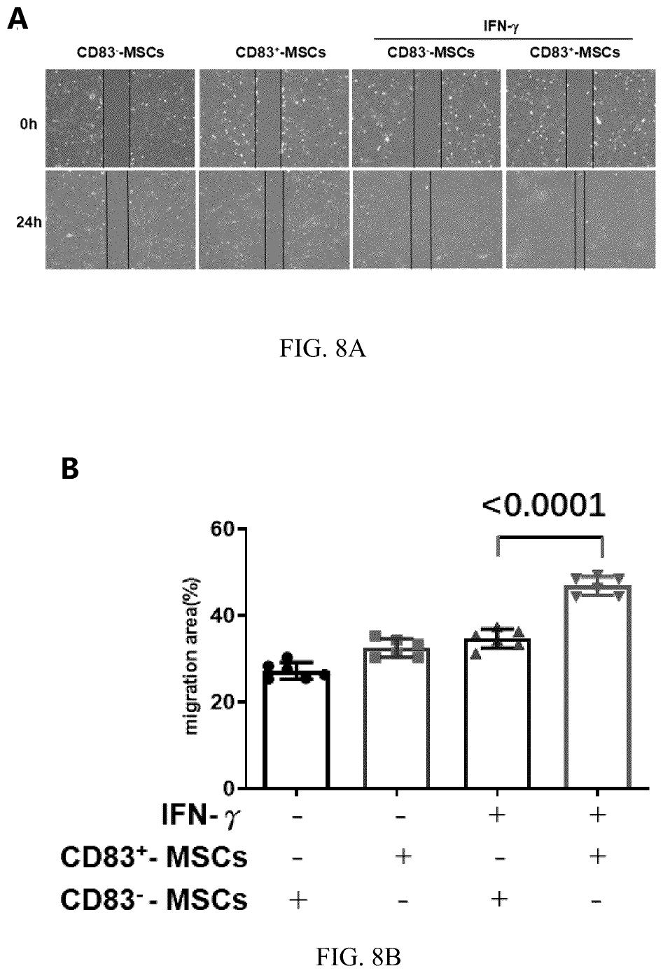

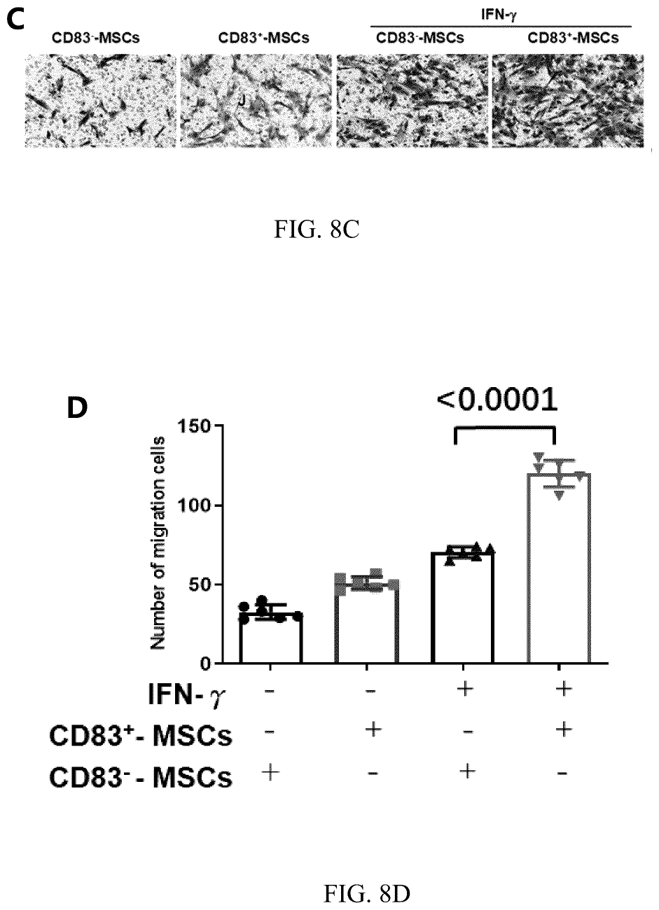

FIG. 8A shows microscopic photographs of cells in the scratch experiment at 0 h and 24 h. FIG. 8B shows the statistical results of scratch area in FIG. 8A. FIG. 8C shows the results of crystal violet staining of the lower layer of the cells after the 24 h Transwell experiment. FIG. 8D shows the statistical result of crystal violet staining positive cells of FIG. 8C. As shown in FIGS. 8A-8D, both the scratching experiment and the Transwell experiment show that there is no statistically significant difference between the two groups under the condition without IFN-γ presence although CD83⁺-MSCs show a better migratory ability compared with CD83⁻-MSCs. However, when the IFN-γ stimulation is added, the migratory ability of CD83⁺-MSCs is significantly enhanced compared to CD83⁻-MSCs.

### Embodiment 3 Induction, Culture and Identification of Immunosuppressive or Anti-Inflammatory Function Enhanced CD83⁺PD-L1⁺ Double Positive MSCs

CD83⁺-MSCs prepared by Embodiment 2 were induced by the IFN-γ alone and/or combined with TNF-α to obtain immunosuppressive or anti-inflammatory function enhanced CD83⁺PD-L1⁺-MSCs. The specific steps were as follows.

### 1. In Vitro Induction Preparation of Immunosuppressive or Anti-Inflammatory Function Enhanced CD83⁺PD-L1⁺ MSCs

The frozen cells of WT-MSCs, CD83⁺-MSCs, and CD83⁻-MSCs obtained in Embodiment 1 and Embodiment 2 were resuscitated and inoculated in T75 culture bottles at a cell density of 4×10⁴ cells/cm², the bottles were placed in an incubator at 37° C. and 5% CO₂, and the cells were cultured to grow to a cell density of greater than or equal to 80%, then the cells were digested with 0.25% TrypLE, and the digested cells were inoculated in T25 culture bottles at a cell density of 4×10⁴ cells/cm² for passaging and expansion culture, and the cells were divided into groups treated individually with factor inducer IFN-γ (0, 5, 10, 20 ng/mL) or TNF-α (0, 5, 10, 20 ng/ml) and groups treated in combination with factor inducers IFN-γ and TNF-α. The culture bottles were incubated at the incubator at 37° C. and 5% CO₂ until the cell density was greater than 80%, and then induced by adding the above factor inducers (IFN-γ (0, 5, 10, 20 ng/ml) and/or TNF-α (0, 5, 10, 20 ng/ml)) for 24 h. The obtained cells were then digested with 0.25% TrypLE, and the cellular samples proteins and mRNA were extracted, and then the Inflammatory protein microarray was used to detect the expression profiles of inflammation-suppressive factors, RT-PCR, immunoblotting assay, and flow cytometry were used to detect the expression of cellular immunosuppressive factor IDO and receptor PD-L1, and a mixed lymphocyte assay was used to assess the proliferation inhibitory function of MSCs on T-lymphocytes, so as to comprehensively assess the immunosuppressive or anti-inflammatory function of CD83⁺-MSCs.

### 2. Inflammatory Protein Microarray to Detect the Expression Profiles of Inflammation-Suppressive Factors in Immunosuppressive or Anti-Inflammatory Function Enhanced CD83⁺PD-L1⁺ MSCs

Protein samples of CD83⁺-MSCs and CD83⁻-MSCs treated with the different stimulation factors mentioned above were extracted; the prepared protein samples were subjected to inflammatory protein microarray assay to compare the expression differences of anti-inflammatory factors of CD83⁺-MSCs and CD83⁻-MSCs after treatment with 20 ng/ml IFN-γ alone.

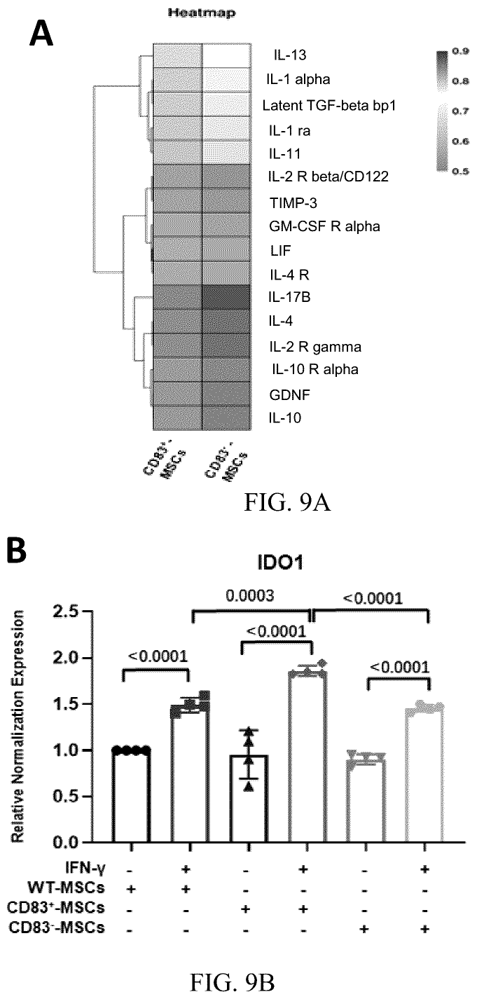

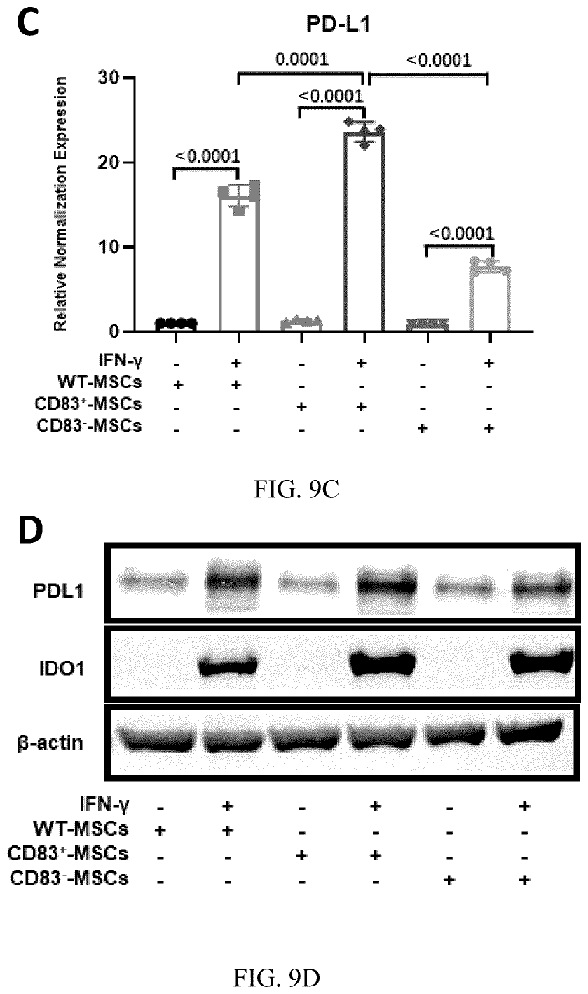

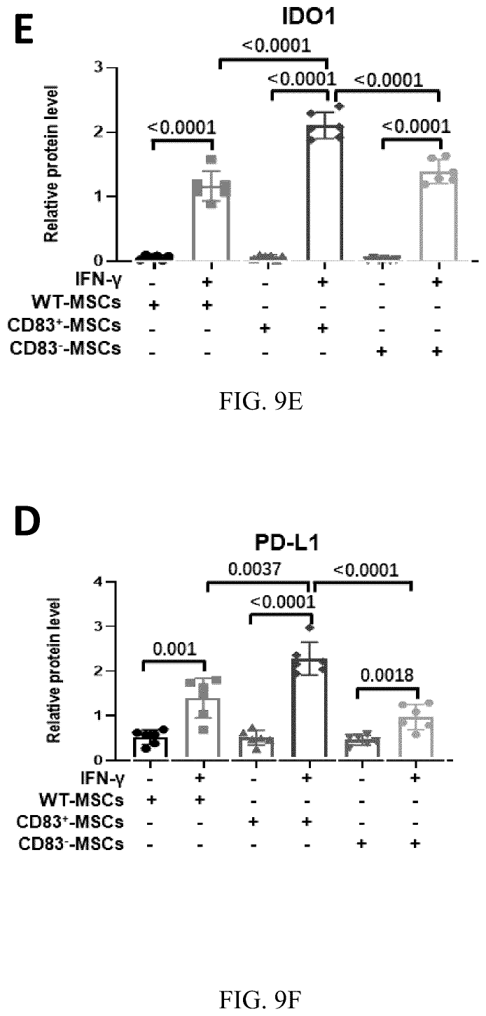

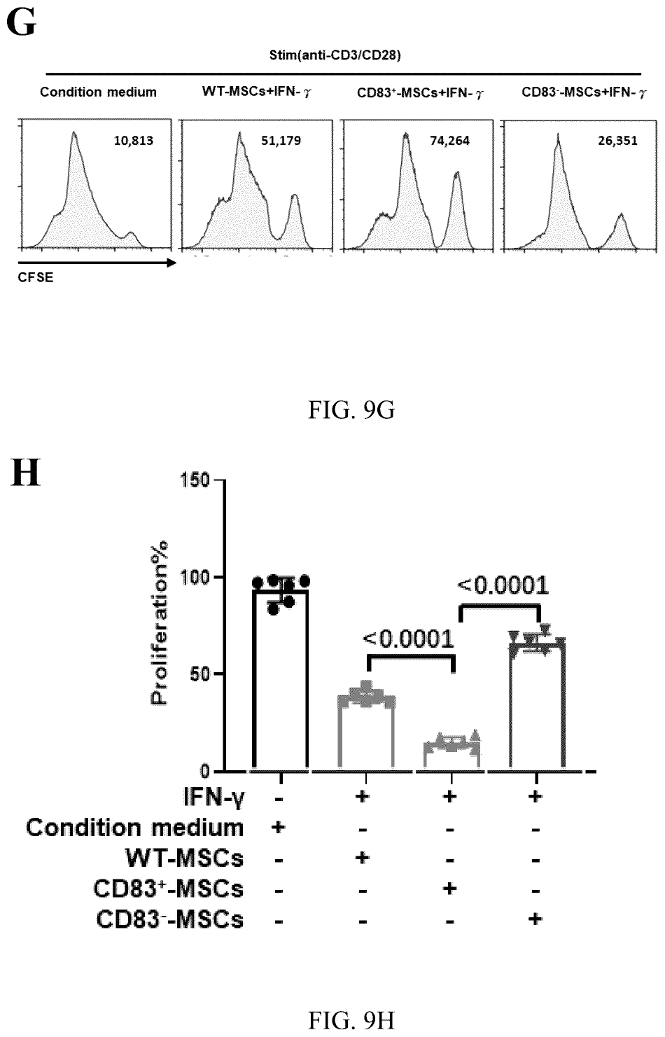

As shown in FIG. 9A, there is significant differences in inflammatory/inflammation-suppressing factor expression between CD83⁺-MSCs and CD83⁻-MSCs cell subpopulations treated with the IFN-γ. In comparison, CD83⁺-MSCs show a higher sensitivity to the regulation of the expression of IFN-γ-mediated inflammatory factors, manifested by a significant increase in the expression of IL-10, GDNF, IL-10Rα, IL-2Rγ, IL-4, IL-4R, IL-17B, LIF, GM-CSFRα, TIMP-3, and IL-2Rβ, suggesting that the subpopulation of CD83⁺-MSCs is more sensitive to IFN-γ-mediated inflammatory function-enhanced of MSCs and can be recognized as an anti-inflammatory function-prone subpopulation.

### 3. Quantitative PCR of mRNA Expression of Immunosuppressive Factor IDO and Immunosuppressive Receptor PD-L1 in Immunosuppressive or Anti-Inflammatory Function Enhanced CD83⁺PD-L1⁺ MSCs Cells

Quantitative PCR was used to detect the mRNA expression of immunosuppressive factor IDO and immunosuppressive receptor PD-L1 in immunosuppressive or anti-inflammatory function-prone MSCS.

FIGS. 9B-9C show the results of quantitative PCR assay of IDO1 and PD-L1 expression in MSCs induced by 20 ng/ml IFN-γ alone. According to FIGS. 9B-9C, quantitative PCR assay results show that for the three groups of cells WT-MSCs, CD83⁻-MSCs, and CD83⁺-MSCs, unstimulated by the IFN-γ, the basal expression of immunosuppressive factor IDO1 and immunosuppressive receptor PD-L1 mRNA is kept at a very low level, with no significant difference between them. However, for the three group cells stimulated by the IFN-γ alone for 24 h, the mRNA expression of IDO1 and PD-L1 is significantly up-regulated in all three groups of cells, and compared with the CD83⁻-MSCs, the CD83⁺-MSCs show the most significant increase in mRNA expression of IDO1 and PD-L1, followed by WT-MSCs.

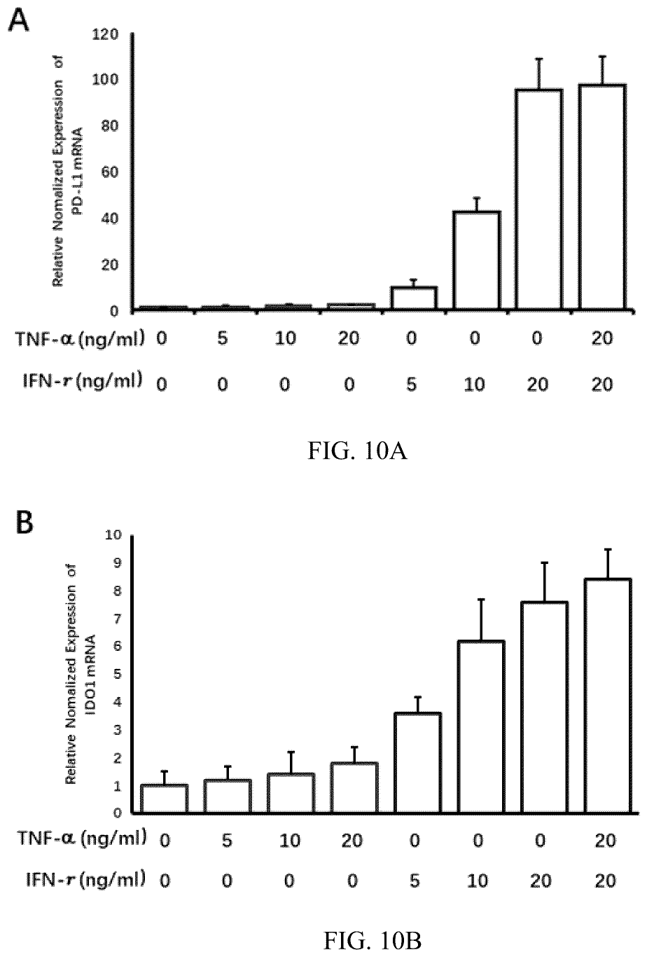

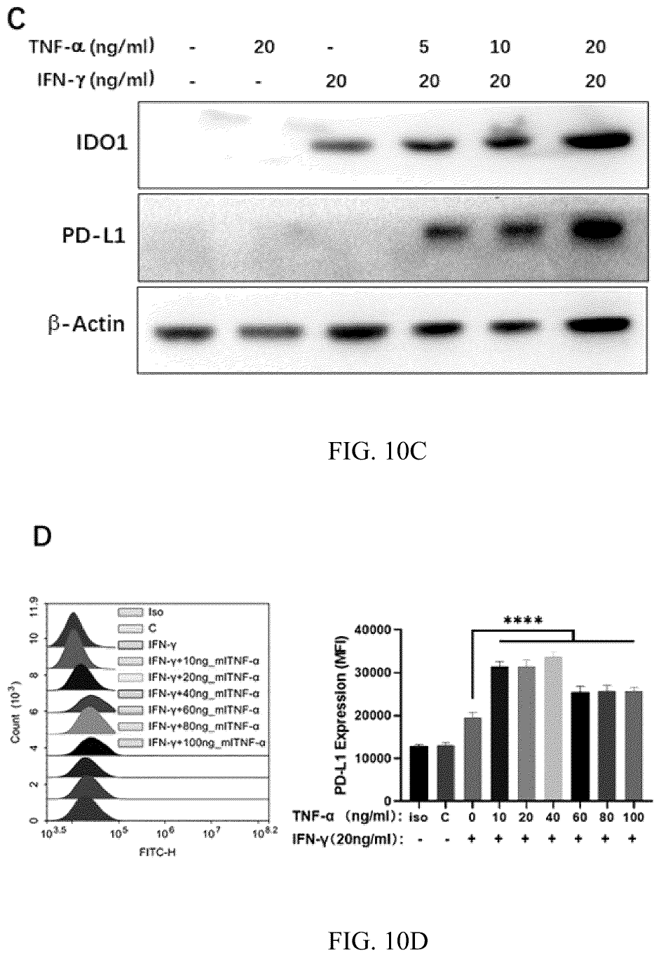

FIGS. 10A-10B show the results of quantitative PCR assays of IDO1 and PD-L1 expression in MSCs induced by different concentrations of the TNF-α and/or combined with the IFN-γ. As shown in the FIGS. 10A-10B, when MSCs are stimulated with 5, 10 and 20 ng/ml of TNF-α alone, it fails to induce the transcription of IDO1 and PD-L1, with only a slight elevation of mRNA. when MSCs are stimulated by 5, 10, and 20 ng/ml of IFN-γ alone, it can induce the transcription of IDO1 and PD-L1, with a significantly elevation of mRNA of IDO1 and PD-L1, and in particular, when MSCs are stimulated by 20 ng/ml of IFN-γ, it can induce to increase mRNA of PD-L1 by up to 85 times. However, different form the results of the immunoblotting assay, the TNF-α in combination with IFN-γ stimulation do not further increase the mRNA expression of IDO1 and PD-L1, on the contrary, 20 ng/ml of TNF-α slightly weakens mRNA expression of IDO1 and PD-L1 induced by IFN-γ.

### 4. Immunoblotting to Detect Protein Expression of IDO and PD-L1 in Immunosuppressive or Anti-Inflammatory Function Enhanced CD83⁺PD-L1⁺ MSCs

The protein samples of WT-MSCs, CD83⁺-MSCs, and CD83⁻-MSCs treated with different stimulation factors mentioned above were extracted; the prepared protein samples were subjected to polyacrylamide gel electrophoresis (SDS-PAGE); and then the electrophoresed gels were analyzed by Western blot.

FIGS. 9D-9F illustrate the immunoblotting assay results of IDO1 and PD-L1 expression in MSCs induced by 20 ng/ml IFN-γ alone, and FIGS. 9E-9F are the results of immunoblotting optical density analyses of IDO1 and PD-L1 (also known as CD274) in FIG. 9D, respectively. Immunoblotting results from FIGS. 9D-9F show that the MSCs stimulated by IFN-γ alone for 24 h can induce both IDO1 and PD-L1 (CD274) protein expression, and CD83⁺-MSCs express IDO1 and PD-L1 more significantly compared to CD83⁻-MSCs.

FIG. 10C shows the results of immunoblotting assay of IDO1 and PD-L1 expression in MSCs induced by different concentrations of the TNF-α combined with the IFN-γ. Immunoblotting results from FIG. 10C show that MSCs stimulated by 20 ng/ml TNF-α alone for 24 h cannot induce IDO1 expression or PD-L1 expression. However, keeping 20 ng/mL IFN-γ combined with different concentrations of TNF-α to treat the MSCs, the results show that the addition of 5 ng/mL TNF-α could induce both IDO1 and PD-L1 expression, and the expression of IDO1 and PD-L1 gradually increase with the increase of TNF-α concentration.

### 5. Flow Cytometry Detection of Immune Checkpoint Receptor PD-L1 Expression in Immunosuppressive or Anti-Inflammatory Function Enhanced CD83⁺PD-L1⁺ MSCs

CD83⁺-MSCs were cultured to 80% of fusion degree, followed by treatment with 20 ng/mL IFN-γ alone or in combination with 20 ng/ml, 40 ng/ml, 60 ng/ml, 80 ng/ml, and 100 ng/mL TNF-α and with PBS for 24 h as a negative control. The cells were digested with 0.25% TrypLE and then washed and resuspended with saline to prepare into single cell suspension (1×10⁶ cells/sample). PD-L1 FITC was added to the cell suspension and incubated for 30 min at room temperature in the dark; and washed 2 times with PBS and then flow assay was performed.

FIG. 10D shows the flow assay results of PD-L1 expression in CD83⁺-MSCs induced by IFN-γ alone or in combination with TNF-α. As shown in FIG. 10D, the flow assay of fluorescence intensity (MFI) of PD-L1 expression of CD83⁺-MSCs increases by about 55% after 24 h of induction by 20 ng/ml IFN-γ alone, whereas the flow assay of fluorescence intensity (MFI) of the PD-L1 expression significantly increases by about 145% after 24 h of induction by 20 ng/mL IFN-γ combined with 10 ng/ml TNF-α, and with the TNF-α concentration increasing to 20 ng/ml and 40 ng/ml, there is a slight increase in the flow assay of fluorescence intensity (MFI) of PD-L1 expression. However, the increased fluorescence intensity of PD-L1 expression is suppressed when the TNF-γ concentration is more than 60 ng/ml, suggesting that the combination of IFN-γ and TNF-α treatment can enhance the expression of the immunosuppressive receptor PD-L1 and the optimal combined concentration of TNF-α ranges from 10 ng/mL-40 ng/mL.

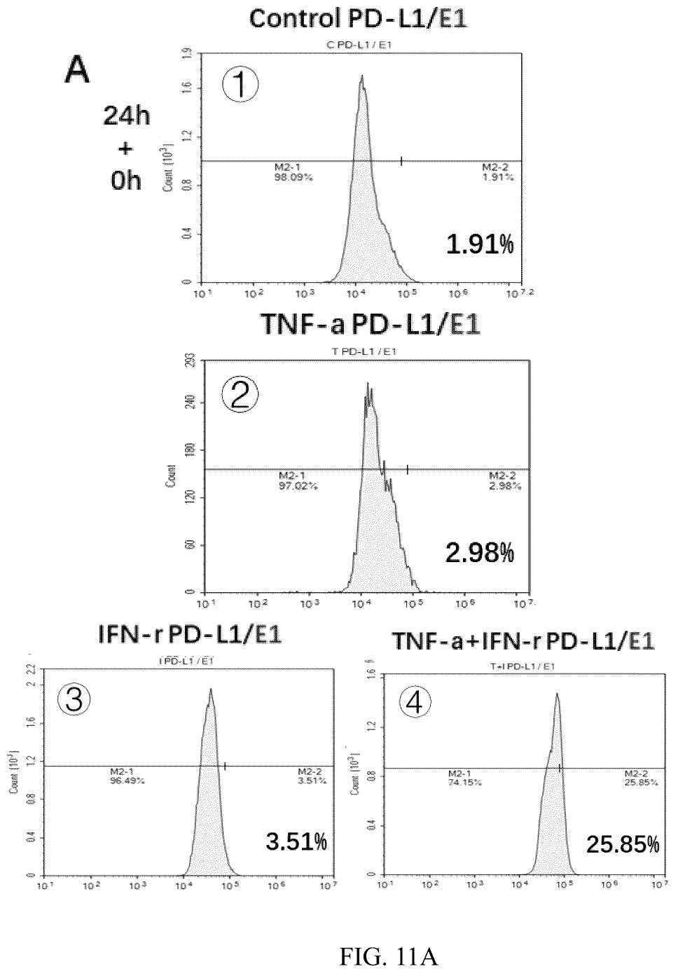

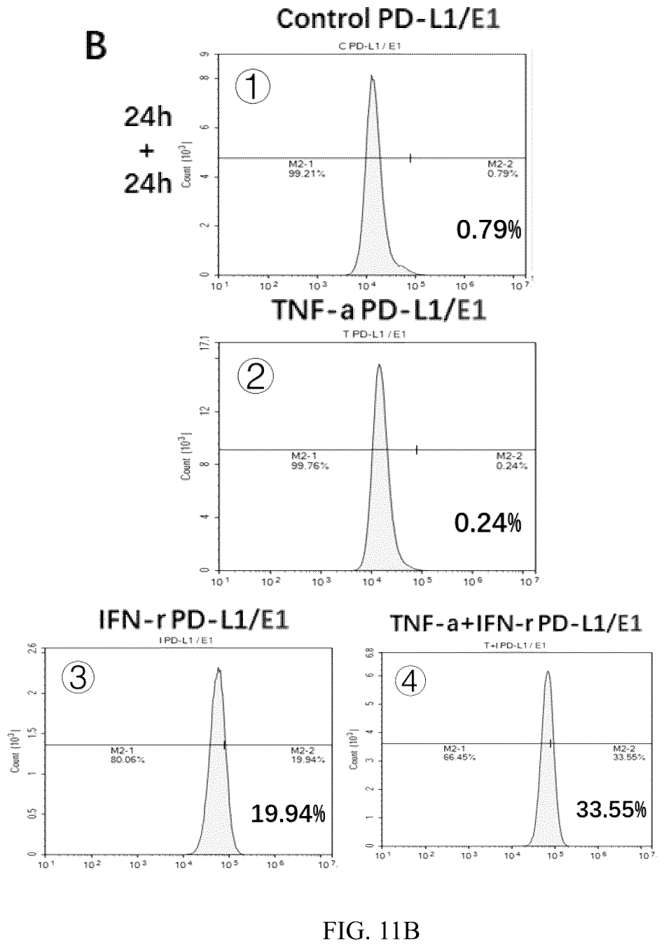

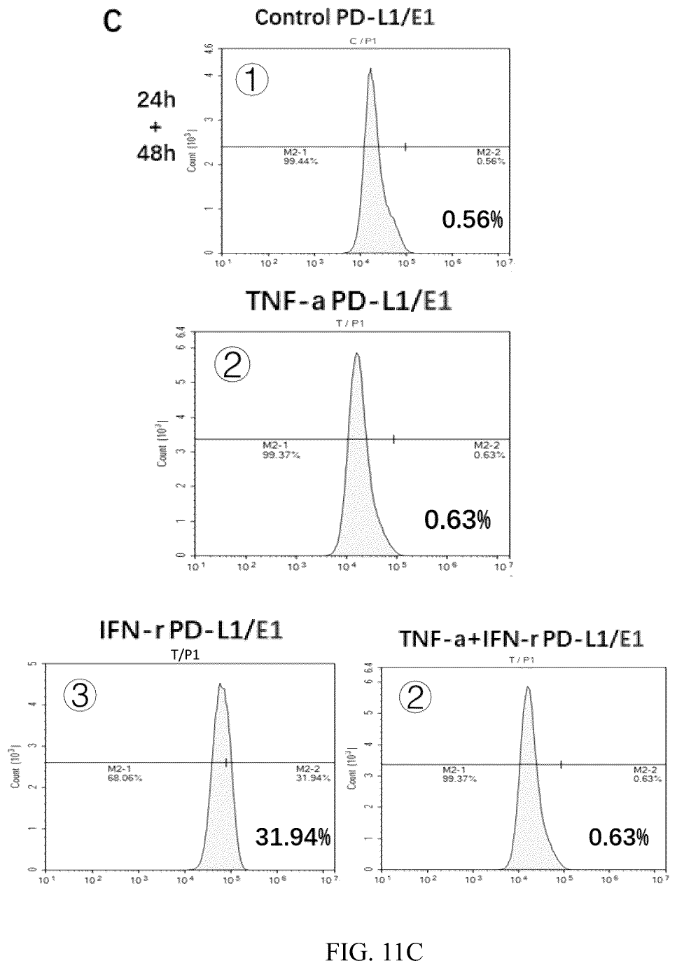

FIG. 11A (1)-(4) illustrates the flow assay results of PD-L1 expression after 24 h induction of CD83⁺-MSCs by no induction factor, 20 ng/ml TNF-α alone, 20 ng/ml IFN-γ alone, and 20 ng/ml TNF-α in combination with 20 ng/ml IFN-γ, respectively. FIG. 11B (1)-(4) illustrates the flow assay results of PD-L1 expression after 24 h induction of CD83⁺-MSCs by no induction factor, 20 ng/mL TNF-α alone, 20 ng/mL IFN-γ alone, and 20 ng/ml TNF-α combined with 20 ng/ml IFN-γ, respectively, followed by 24 h culture of MSCs after withdrawing the stimulating factors. FIG. 11C (1)-(4) illustrates the flow assay results of PD-L1 expression after 24 h induction of CD83⁺-MSCs cells by no inducing factor, 20 ng/ml TNF-α alone, 20 ng/ml IFN-γ alone, and 20 ng/ml TNF-α combined with 20 ng/ml IFN-γ, respectively, followed by 48 h culture after withdrawing the stimulating factor. As shown in FIGS. 11A-11C, for the CD83⁺-MSCs induced by 20 ng/ml TNF-α alone, the expression of the immunosuppressive receptor PD-L1 is not affected across all tested conditions: 24 h induction, 24 h induction followed by 24 h culture, and 24 h induction followed by 48 h culture. In contrast, for the CD83⁺-MSCs induced by 20 ng/ml IFN-γ alone, there is a slight increase in the expression of the immunosuppressive receptor PD-L1 after 24 h stimulation, but there is significantly increase in the expression of PD-L1 after 24 h stimulation followed by 24 h or 48 h culture, the expression of PD-L1 reaching 31.94% after 24 h stimulation followed by 48 h culture. In comparison, for CD83⁺-MSCs induced by 20 ng/mL TNF-α and 20 ng/ml IFN-γ, there is a significantly and rapidly increase in the expression of PD-L1, the positive expression rate of PD-L1 rapidly reaches 25.85% at 24 h induction, and the positive expression rate reaches 33.55% and 61.23% respectively, after 24 h induction followed by 24 h or 48 h culture, which is almost twice of that of CD83⁺-MSCs induced by IFN-γ alone.

### 6. Flow Cytometry Detection of the Proliferation Inhibition Effect of Immunosuppressive or Anti-Inflammatory Enhanced CD83⁺PD-L1⁺ MSCs on PBMC Cells

Nuclear staining was performed in 1×10⁷ peripheral blood mononuclear cells (PBMCs) by adding 1 μL (1000×) of CFSE fluorescent dye, incubated in the dark for 20 min, and washed twice with PBS. PBMCs cells without CFSE staining were used as a negative control, and non co-cultured PBMCs cells were stained and used as a positive control. After that, the stained PBMCs cells were respectively co-cultured with three kinds of CD83⁻-MSCs, CD83⁺-MSCs, and WT-MSCs that were treated by 20 ng/mL IFN-γ for 24 h, respectively, and IL-2 was added to each well of the co-culture system at a final concentration of 100 IU/mL. The changes in CFSE fluorescence values of each group of PBMCs were detected by flow assay after 3 days of co-culturing to determine the inhibitory effect on the proliferation of PBMCs. FIG. 9G shows the intracellular CFSE fluorescence changes of PBMCs co-cultured with MSCs by flow cytometry, and FIG. 9H shows the inhibition rate of cell proliferation of PBMCs calculated from FIG. 9G. As shown in FIGS. 9G-9H, all three types of MSCs (CD83⁻-MSCs, CD83⁺-MSCs, and WT-MSCs treated with IFN-γ) significantly inhibits the proliferation of PBMCs. In comparison, CD83⁺-MSCs has the strongest inhibitory effect on the proliferation of PBMCs, followed by WT-MSCs, with the CD83⁻-MSCs having the worst inhibitory effect. Embodiment 4 Use and efficacy evaluation of immunosuppressive or anti-inflammatory function-prone CD83⁺ human umbilical cord MSCs in the treatment of mice with collagen induced arthritis

### 1. Establishment of Collagen Induced Arthritis (CIA) Mice Model and MSCs Therapeutic Protocols and Procedures

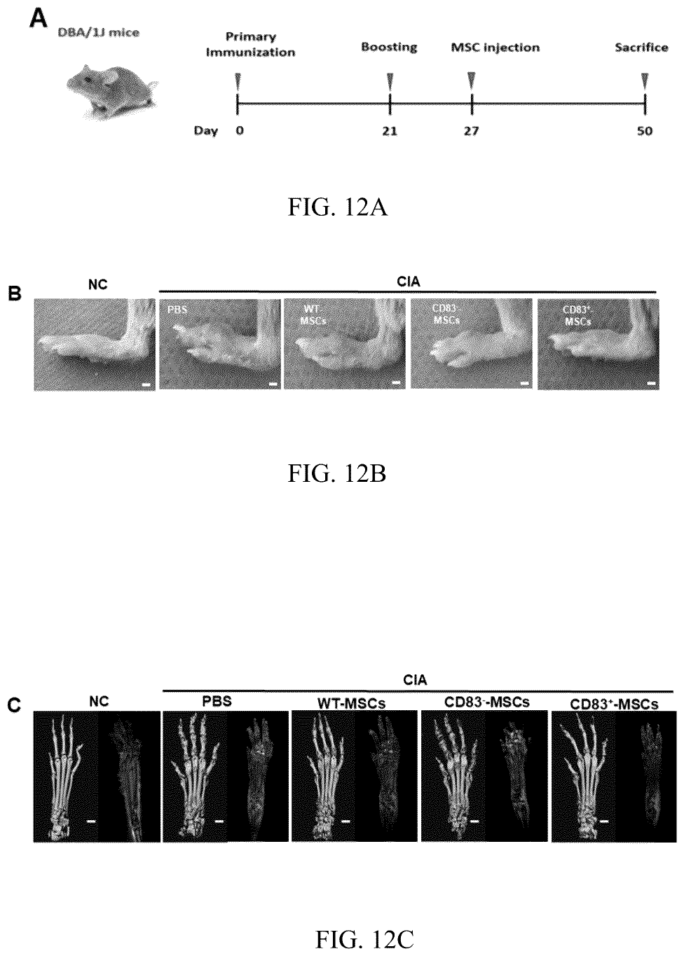

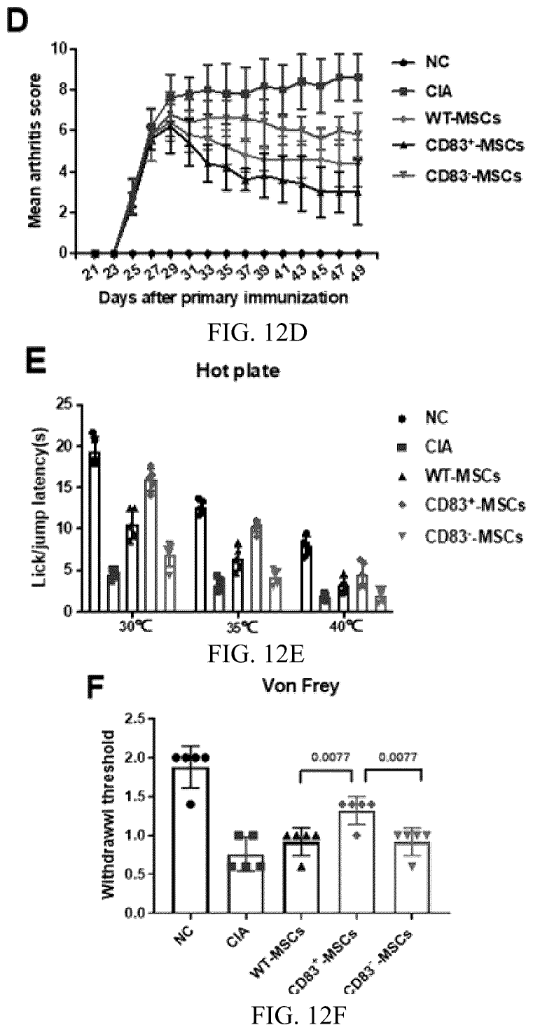

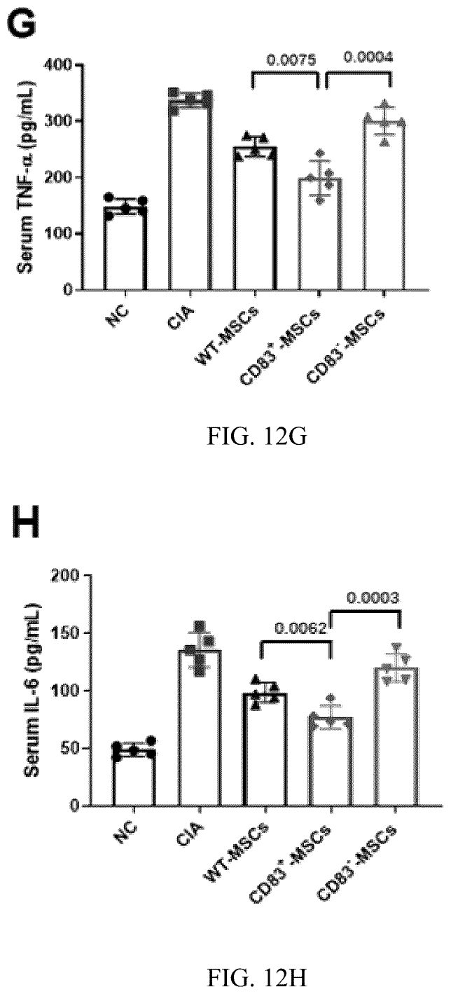

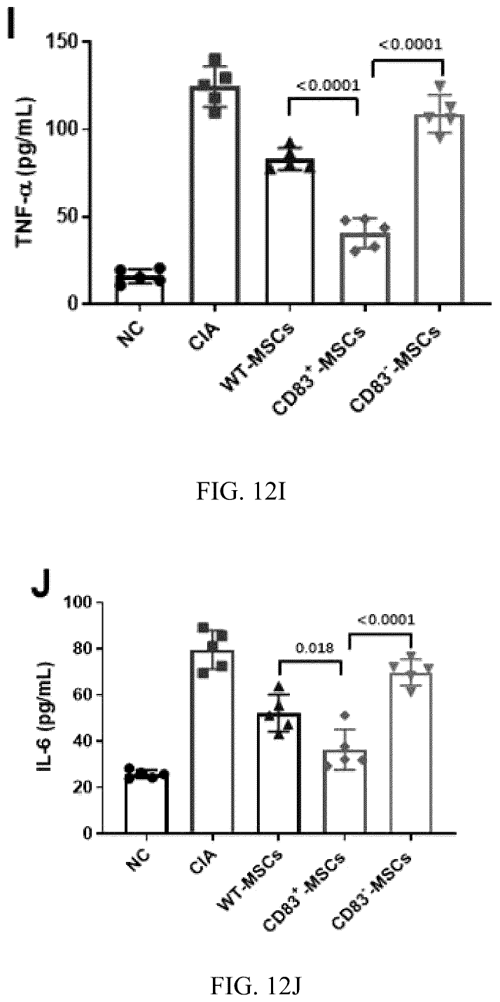

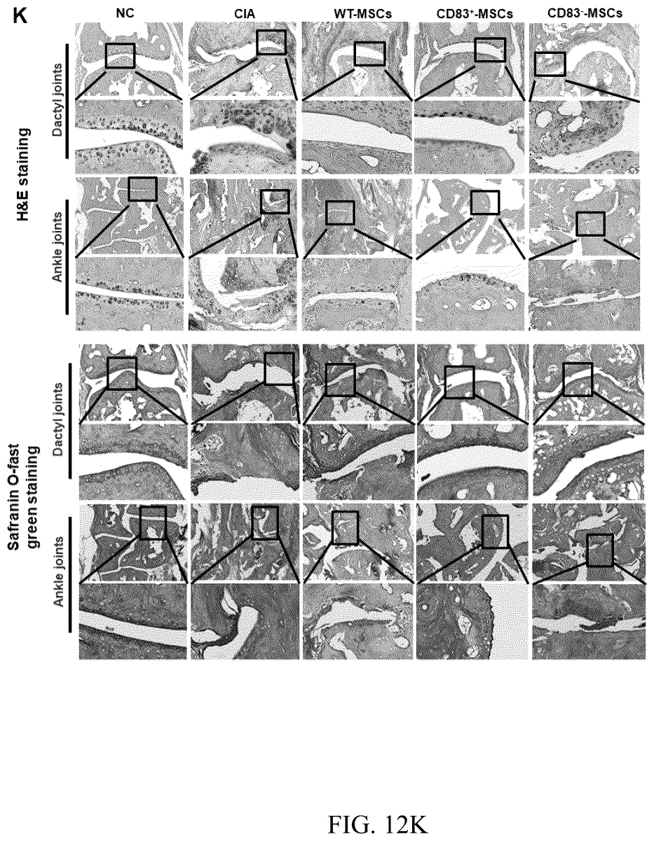

CIA mice model was constructed according to the prior literature, which can reduce the individual differences of the animals to better evaluate the therapeutic efficacy. When an arthritis score reached or exceeded 3 points after a booster injection of collagen, i.e., treatment with MSCs was initiated again after onset. Meanwhile, WT-MSCs, CD83⁺-MSCs, and CD83⁻-MSCs seed cells obtained by culturing or sorting according to Embodiments 1 and 2 were resuscitated and cultured for 48 h until cell fusion reached 80%, cells were harvested to prepare MSCs cell preparations. The mice models were treated by single tail vein infusion of 1×10⁶ wild-type human umbilical cord MSCs (WT-MSCs group, the treatment control group), CD83⁺-MSCs, CD83⁻-MSCs, and the PBS group (the non-treatment control group), as shown in the procedure in FIG. 12A. The CIA mice models selected for the present disclosure were subjected to tail vein injection of MSCs after the onset of arthritis (approximately 3-5 days after the reinforcing collagen injection). Pain thresholds for thermal and mechanical pricking were tested in mice on day 50. Additionally, mice were euthanized on day 50, and blood and limb joints were taken for flow cytometry analysis, cytokine detection, and histopathology examination, along with imaging evaluation.

Clinical scoring protocol for arthritis in the CIA mice model: arthritis clinical scoring was assessed in the CIA mice using foot-plantar swelling. Clinical arthritis was assessed using the following scores: 0, no swelling; 1, mild swelling and erythema; 2, marked edema; and 3, joint stiffness. Scores were summarized for each limb, with a maximum possible score of 12 per mice.

### 2. Evaluation of the Efficacy of Immunosuppressive or Anti-Inflammatory Function-Prone CD83⁺-MSCs in a Mice Model with Collagen Induced Arthritis

(1) Observations and Swelling Scores of CIA Mice with Arthritis Treated with MSCs

Swelling of each limb was scored in each treatment group of mice according to the arthritis clinical scoring criteria for foot-plantar swelling. FIG. 12B (General observation) and FIG. 12D (Swelling scoring) show the results of arthritis swelling assessment in mice of the negative control (NC) and CIA mice injected with PBS, WT-MSCs, CD83⁺-MSCs, and CD83⁻-MSCs intravenously. The CIA mice model was successfully established after 25 days of collagen induction, and CIA mice were administered by MSCs injection on day 27, followed by scoring of joint swelling. The results show that all three types of MSCs (WT-MSCs, CD83⁺-MSCs, and CD83-MSCs) significantly improve the swelling of toes in CIA mice, and in comparison, CD83⁺-MSCs have the most significant anti-edematous effect on CIA mice, with the lowest index score.

(2) Imaging Evaluation of CIA Mice with Arthritis Treated with MSCs

At the end of the experiment (on day 50), vivaCT 40 small animal CT (SCANO MEDICAL, Switzerland) and Bruker BioSpec 7T/20 cm system small animal Magnetic Resonance Imaging (MRI) (Bruker, Germany) were used on the hind limbs of CIA mice for imaging examination.

FIG. 12C shows the imaging results of mice of negative control (NC) and CIA mice injected intravenously with PBS, WT-MSCs, CD83⁺-MSCs, and CD83⁻-MSCs. Representative images of the hind limbs of CIA mice were imaged on day 50 using small animal MRI and small animal CT. At least 5 mice were used in each group: NC=Negative control (n=5 mice), PBS=CIA mice PBS-treated group (n=10 mice), WT-MSCs=CIA mice WT-MSCs-treated group (n=10 mice), CD83⁺-MSCs=CIA mice CD83⁺-MSCs-treated group (n=10 mice), CD83⁻-MSCs=CIA mice CD83⁻-MSCs treatment group (n=10 mice), and all results were presented as mean+standard deviation. As shown in FIG. 12B (results of small animal CT test) and FIG. 12C (results of small animal MRI test), compared with the PBS-treated control, single intravenous injection of all three types of MSCs (WT-MSCs, CD83⁺-MSCs, and CD83-MSCs) significantly improves synovitis, arthritis, cartilage damage, and bone destruction in the CIA mice. In comparison, the CD83⁺-MSCs has the best therapeutic efficacy, followed by the WT-MSCs, and CD83⁻-MSCs has the worst therapeutic efficacy.

(3) Evaluation of Toes Pain Threshold of CIA Mice with Arthritic Treated with MSCs

At the end of the experiment (on day 50), thermal injurious response was assessed with a hot plate pain meter to obtain thermal pain thresholds of CIA mice, and mechanical stabbing pain thresholds of CIA mice were obtained by measuring the thresholds of the foot reduction response on the plantar surface of the hind foot using the Von Frey fiber filament applied pressure manner.

FIGS. 12E-12F illustrate the examination results of thermal and mechanical pain threshold in negative control mice (NC) and CIA mice injected intravenously with PBS, WT-MSCs, CD83⁺-MSCs, and CD83⁻-MSCs, respectively. As shown in FIG. 12E (thermal pain threshold) and FIG. 12F (mechanical pain threshold), compared with the PBS-treated control, single intravenous injection of all three types of MSCs (WT-MSCs, CD83⁺-MSCs, and CD83-MSCs) significantly improves thermal and mechanical pain thresholds of CIA mice. In comparison, the CD83⁺-MSCs show the best improvement in pain thresholds, and the CD83⁻-MSCs show the worst improvement in pain thresholds.

(4) Evaluation of Systemic and Localized Toe Inflammation in CIA Mice with Arthritic Treated with MSCs

At the end of the experiment (on day 50), mice were euthanized, serum and local soft tissues of toes were taken, and the levels of TNF-α and IL-6 in serum and soft tissues were detected by ELISA to determine the systemic and local anti-inflammatory effects of MSCs on CIA mice.

FIGS. 12G-12H show the ELISA results of serum inflammatory factors TNF-α and IL-6 in CIA mice treated with the three cells on day 50, respectively; and FIGS. 12I-12J show the ELISA results of localized tissue inflammatory factors TNF-α and IL-6 in the limbs, feet, and metatarsals of CIA mice treated with the three cells on day 50, respectively. As shown in FIGS. 12G-12J, compared with the negative control, both serum and soft tissue local inflammatory factors TNF-α and IL-6 are significantly elevated in CIA mice. Compared with the PBS-treated group, after single treatment with the three types of MSCs (WT-MSCs, CD83⁺-MSCs, and CD83-MSCs), the serum and soft tissue local inflammatory factors TNF-α and IL-6 levels are significantly reduced. In comparison, there is the most obvious anti-inflammatory effect in the CD83⁺-MSCs group and the worst anti-inflammatory effect in the CD83⁻-MSCs group.

(5) Histopathological Evaluation of CIA Mice with Arthritis Treated with MSCs

On day 50 of the experiment, all mice were euthanized and limb joint tissues were taken from mice; limb specimens fixed with 4% paraformaldehyde were decalcified with a decalcification solution containing 15% EDTA, and then the decalcified limbs were dehydrated and paraffin-embedded according to standard histological manners.

Hematoxylin-eosin staining (H&E): after routine xylene dewaxing and gradient alcohol hydration of the slices, they were put into warm hematoxylin staining solution for 2 min, and the staining solution was quickly rinsed out by water, and then rinsed again by water after being hydrated using 1% hydrochloric acid in alcohol for 15 s, then staining effect was observed under the microscope, and the criterion was that nuclei were bluish-purple, and the intercellular matrix was not stained. Then the slices were stained with eosin for 1 min, followed by gradient alcohol dehydration, xylene transparency, neutral gum sealing. The severity of arthritis was assessed by H&E staining: normal synovium, 0 points; synovial hypertrophy and cellular infiltration, 1 point; vascular membrane and cartilage erosion, 2 points; cartilage and subchondral bone erosion, 3 points; and loss of joint integrity and ankylosis, 4 points. Evaluations were performed by multiple third-party testers, and the average of scores of multiple testers was used as the final value.

Safranin O and Fast Green staining: the slices were dewaxed and rehydrated to water, 0.02% Fast Green for 5 min (not rinsing), 1% acetic acid for 30 s (not rinsing), 0.1% Safranin O for 20 min (not rinsing), rinsed for 2 min in 95% ethanol, then rinsed twice for 3 min with 100% alcohol, and xylene twice for 2 min, and covered with coverslips. Slices were stained with Safranin O and Fast Green to evaluate cartilage destruction.

FIG. 12K shows the histopathological evaluation results of mice euthanized on day 50, paraffin-embedded slices of metacarpal and phalangeal posterior joints of CIA mice were stained with H&E, Safranin O, and Fast Green, respectively to evaluate their histological joint damage and cartilage destruction, and representative images were selected, with a scale of 200 μm. At least 5 mice were included in each group: in FIG. 12K, NC is the negative control (n=5 mice), PBS is the PBS-treated group of CIA mice (n=10 mice), WT-MSCs is the WT-MSCs treatment group of CIA mice (n=10 mice), and CD83⁺-MSCs is the CD83⁺-MSCs treatment group of CIA mice (n=10 mice), CD83⁻-MSCs is the CD83⁻-MSCs treatment group of CIA mice (n=10 mice). As shown in FIG. 12K, compared with the PBS-treated group, all three kinds of MSCs (WT-MSCs, CD83⁺-MSCs, and CD83-MSCs) can significantly reduce synovitis, joint destruction and cartilage destruction in experimental arthritis. In comparison, the CD83⁺-MSCs group has the most obvious improvement effect on osteoarthritis, CD83⁻-MSCs group has the worst improvement effect on osteoarthritis. Meanwhile, none of the mice treated with WT-MSCs, CD83⁺-MSCs, and CD83⁻-MSCs shows any side effects or death before the termination of the experiment.

### Embodiment 5 Use and Efficacy Evaluation of Immunosuppressive or Anti-Inflammatory Function-Prone Artificial CD83 Overexpressed MSCs in the Treatment of Mice with Collagen Induced Arthritis

### 1. Establishment of Collagen Induced Arthritis (CIA) Mice Model and MSCs Treatment Protocols and Procedures

CIA mice model was established according to Embodiment 4, and the preparation of artificial CD83 overexpressed MSCs was performed according to Embodiment 2, i.e., wild-type human umbilical cord MSCs (WT-MSCs) obtained in Embodiment 1 and CD83⁺ human umbilical cord MSCs (CD83⁺-MSCs) obtained by screening in Embodiment 2 were used as the seed cells, respectively. On the basis, lentiviral overexpression technology was used to obtain CD83 overexpressed WT^{CD83 OE}-MSCs and CD83^{+/OE}-MSCs. The therapeutic efficacy of artificial CD83 overexpressed MSCs on arthritis in CIA mice was later evaluated according to the MSCs treatment protocol and procedures in Embodiment 4. The animals were grouped into a negative control group of normal mice (NC) and CIA mice injected intravenously with PBS, CD83^{+/GFP}-MSCs, CD83^{+/OE}-MSCs, and WT^{CD83 OE}-MSCs.

### 2. Efficacy Evaluation of Immunosuppressive or Anti-Inflammatory Function-Prone Artificial CD83 Overexpressed MSCs in the Treatment of CIA Mice with Arthritis

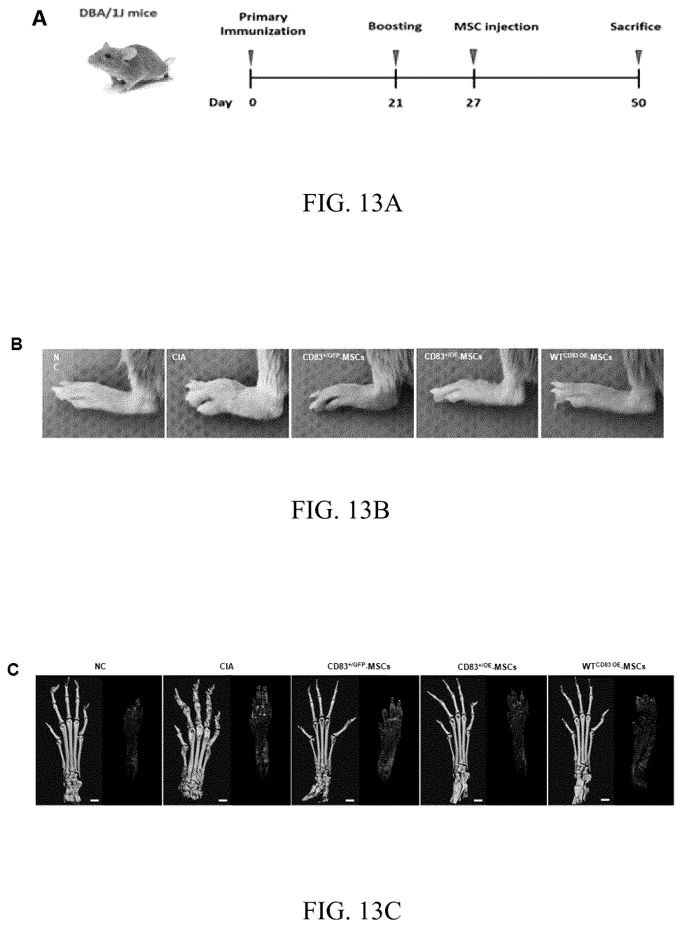

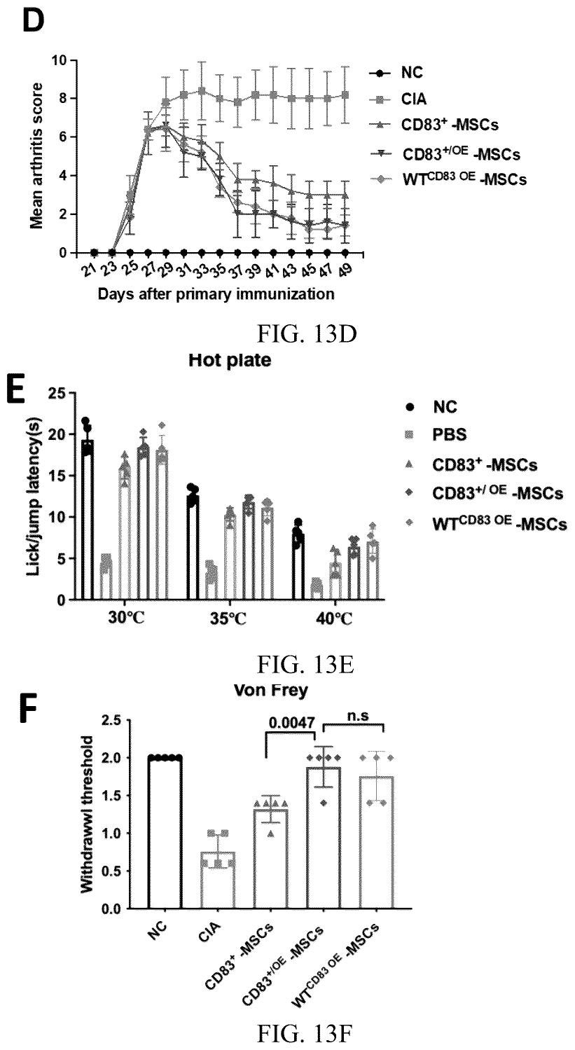

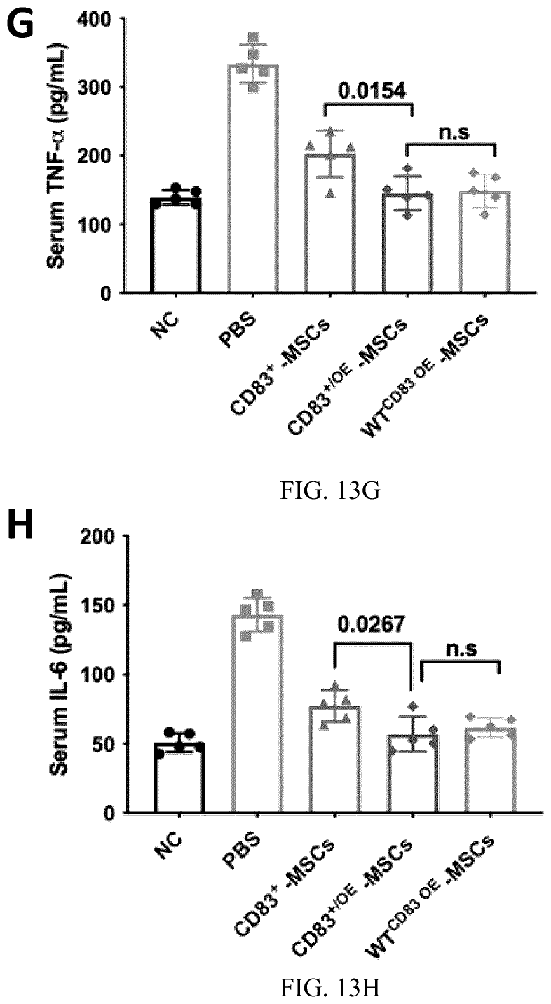

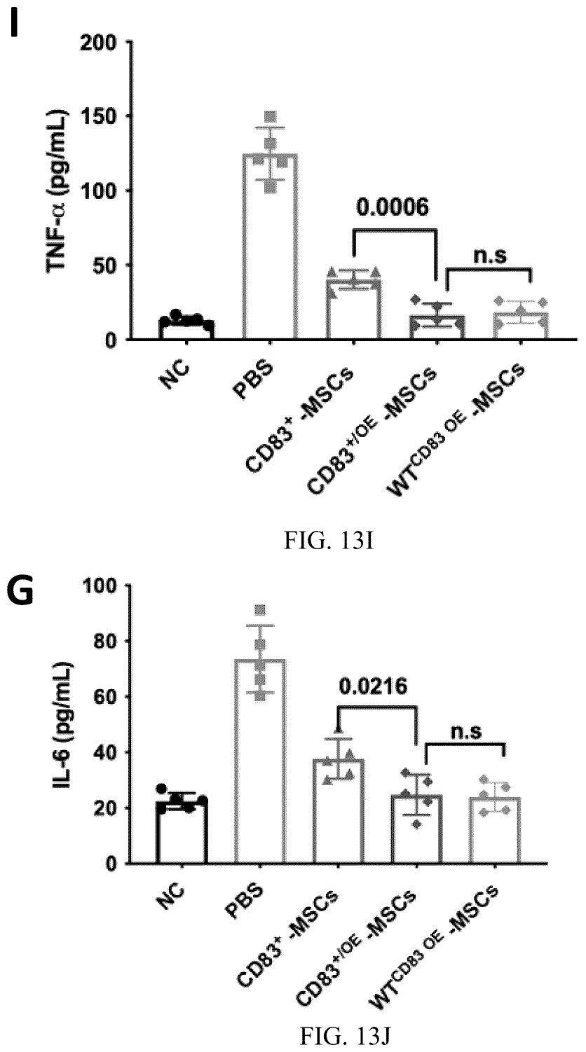

The efficacy evaluation included gross observation and swelling score of arthritis, pain threshold evaluation, systemic and localized toe inflammation evaluation, imaging and pathology evaluation, see Embodiment 4 for specific evaluation manners and measures. FIGS. 13A-13J illustrate the evaluation of the therapeutic efficacy of artificial CD83 overexpressed MSCs on CIA mice with arthritis. FIG. 13A shows the process of treating CIA mice with arthritis using artificial CD83 overexpressed MSCs; and FIGS. 13B and 13D show the observation of gross erythema and swelling of the toe joints and swelling scores of CIA mice with arthritic treated with artificial CD83 overexpressed MSCs. It can be seen that compared to the PBS-treated group, the results show that CD83^{+/GFP}-MSCs, CD83^{+/OE}-MSCs, and WT^{CD83 OE}-MSCs have therapeutic efficacy on mice with arthritis, with significant improvement in joint erythema and scores, CD83 overexpressed CD83^{+/OE}-MSCs and WT^{CD83 OE}-MSCs have the most significant improvement in joint erythema and scores, with no significant difference between the two groups. FIG. 13C shows the results of CT and MRI evaluation of the toe joints of CIA mice with arthritic treated with artificial CD83 overexpressed MSCs. The results show that compared with the PBS-treated group, the three groups of MSCs cells (CD83^{+/GFP}-MSCs, CD83^{+/OE}-MSCs, and WT^{CD83 OE}-MSCs) have significant ameliorative effects on synovitis, arthritis, cartilage injury, and bone destruction, the two groups of CD83 overexpressed CD83^{+/OE}-MSCs and WT^{CD83 OE}-MSCs have the most obvious therapeutic efficacy on arthritis, and there is no significant difference between the two groups. FIGS. 13E and 13F show the evaluation results of the thermal and mechanical pain threshold of CIA mice treated with the artificial CD83 overexpressing MSCs. All three groups of cells (CD83^{+/GFP}-MSCs, CD83^{+/OE}-MSCs, and WT^{CD83 OE}-MSCs) can increase the pain threshold of CIA mice, and the two groups of CD83 overexpressed CD83^{+/OE}-MSCs and WT^{CD83 OE}-MSCs have the most obvious improvement on the pain threshold of CIA mice, and there is no significant difference between the two groups. FIGS. 13G and 13H show the ELISA results of serum inflammatory factors TNF-α and IL-6 in CIA mice treated with artificial CD83 overexpressed MSCs on day 50, respectively; and FIGS. 131 and 13J show the ELISA results of inflammatory factors TNF-α and IL-6 in localized tissues of the limbs, feet, and metatarsals of CIA mice treated with artificial CD83 overexpressed MSCs on day 50, respectively. As shown in FIGS. 13G-13J, compared with the negative control, both serum and soft tissue local inflammatory factors TNF-α and IL-6 are significantly increased in CIA mice, and compared with the PBS-treated group, after single treatment with three groups of MSCs (CD83^{+/GFP}-MSCs, CD83^{+/OE}-MSCs, and WT^{CD83 OE}-MSCs), the levels of serum and soft tissue local inflammatory factors TNF-α and IL-6 in CIA mice are significantly reduced. In comparison, the CD83 overexpressed CD83^{+/OE}-MSCs and WT^{CD83 OE}-MSCs groups have the most significant anti-inflammatory effect, and there is no significant difference between the two groups.

### Embodiment 6 Use and Efficacy Evaluation of Immunosuppressive or Anti-Inflammatory Function-Prone CD83⁺ Human Umbilical Cord MSCs in the Treatment of Mice with CLP Sepsis

### 1. Establishment of CLP Sepsis Mice Model and MSCs Treatment Protocols and Procedures

8-week-old BALB/c mice with a weight of approximately 22-25 g were selected for cecum ligation perforation (CLP), and at 3 h postoperatively, sham operation control (sham) and PBS-treated control group were injected with 0.2 mL of sterile PBS via the tail vein, and WT-MSCs, CD83⁺-MSCs, CD83^{+/OE}-MSCs, and WT^{CD83 OE}-MSCs groups were each injected with 200 μL of cell suspension containing 2×10⁵ cells, and antibiotics of imipenem and cilastatin sodium (14 mg/kg) were added after 24 h, the mortality rate of each group was finally counted at 168 h. Serum samples for liver function, renal function, cardiac function and systemic inflammatory index examination, and tissue samples for pathological examination were taken at 72 h postoperatively, and the sample collection procedure was as follows: at 3 h after cecum ligation and perforation (CLP) was performed in BALB/c mice, the sham operation control group and the PBS-treated control group were injected with 0.2 mL of sterile PBS via the tail vein, and the WT-MSCs, and CD83⁺-MSCs, CD83^{+/OE}-MSCs, and WT^{CD83 OE}-MSCs groups were each injected with 200 μL of cell suspension containing 2×10⁵ cells, and antibiotics were added with imipenem and cilastatin sodium (14 mg/kg) after 24 h, at 72 h after CLP, mice in each group were anesthetized with 1% sodium pentobarbital intraperitoneally (40 mg/kg), then the eyeballs were removed to obtain blood, and tissues of important organs were dissected and removed. Whole blood was allowed to stand at room temperature for 2 h, after blood coagulation and stratification, the blood was centrifuged at 1500 r/min for 10 min, and centrifuged for a total of 3 times, serum was collected and tested for the levels of the inflammatory factors TNF-α, IL-1B, and IL-6 by ELISA, and the liver function (including glutamic oxaloacetic aminotransferase AST and glutamic alanine aminotransferase ALT) was tested for CLP mice by the conventional biochemical assay methods, renal function (including serum creatinine, blood urea nitrogen and amylase AMS), and cardiac function (including lactate dehydrogenase LDH, α-hydroxybutyrate dehydrogenase α-HBDH and creatine kinase CK). Tissue specimens were processed according to the pathological tissue examination protocol in Embodiment 4, followed by H&E staining for histopathology.

The preparation process of MSCs used in the Embodiment 6 referred to the Methods and Procedures of Embodiment 5, with the difference that at 48 h before harvesting, the MSCs of each group were treated by addition of 20 ng/ml of IFN-γ and 20 ng/mL of TNF-α for PD-L1 positive expression of inflammation suppression empowerment stimulation for 24 h according to the protocol in Embodiment 3, and then cultured in the medium without IFN-γ and TNF-α for 24 h to obtain the cells for subsequent sepsis mouse treatment experiments.

### 2. Protective Effect of Immunosuppressive or Anti-Inflammatory Function-Prone CD83⁺ Human Umbilical Cord MSCs on Survival of CLP Sepsis Mice

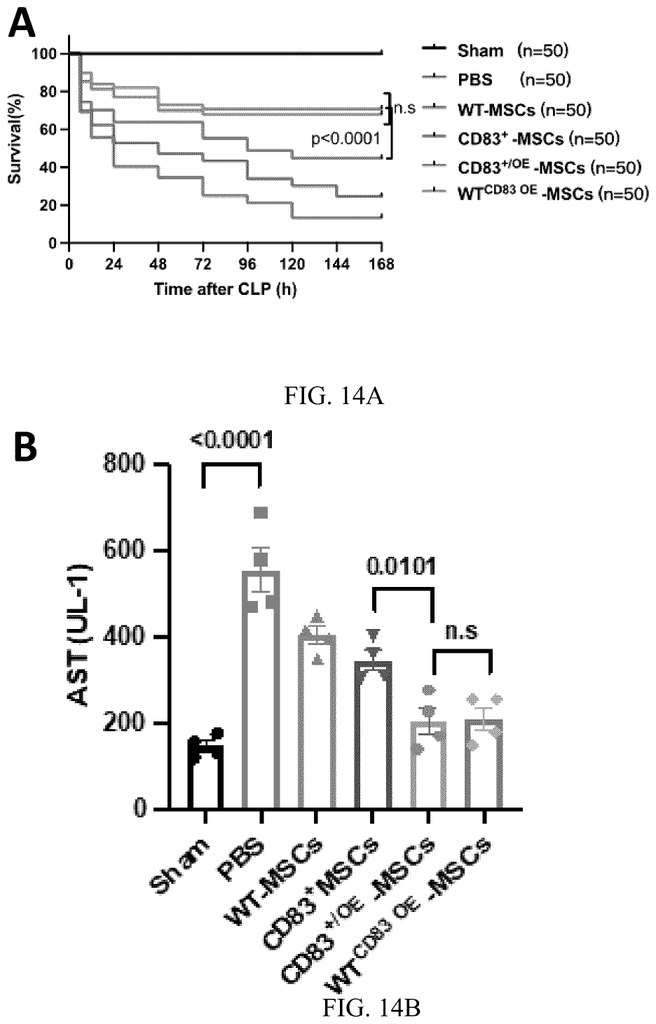

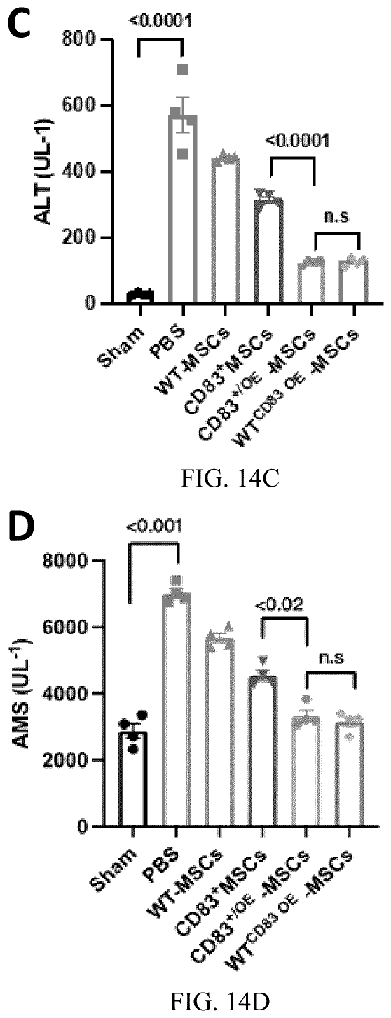

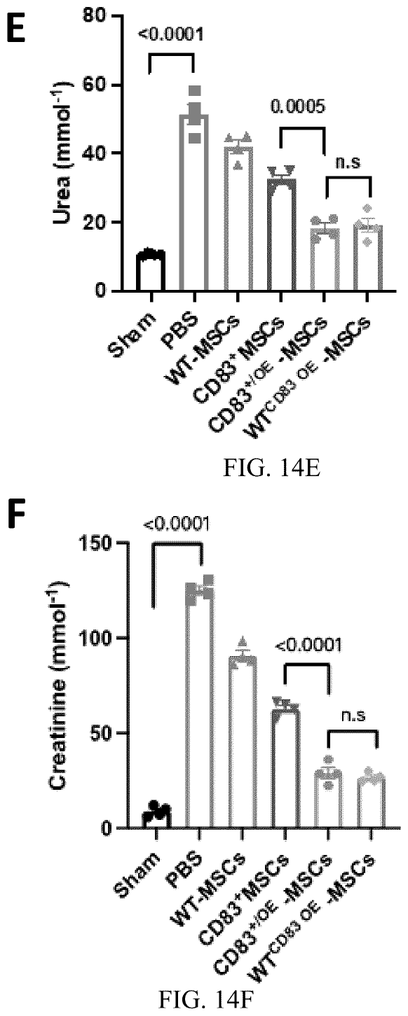

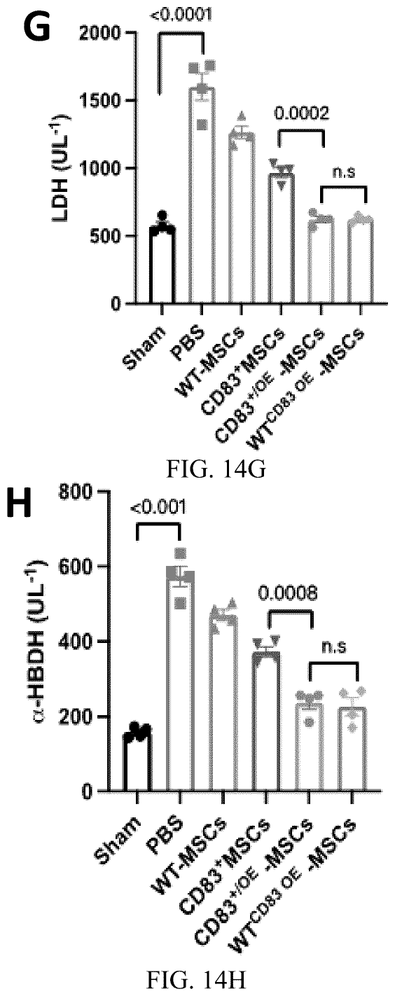

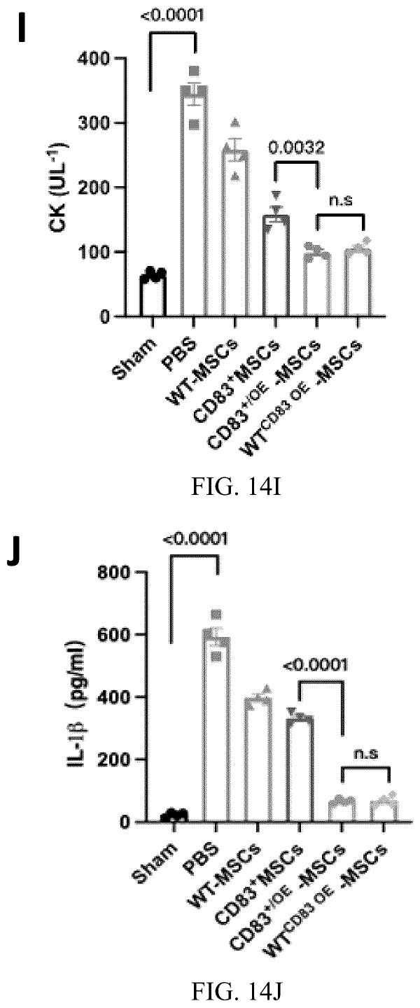

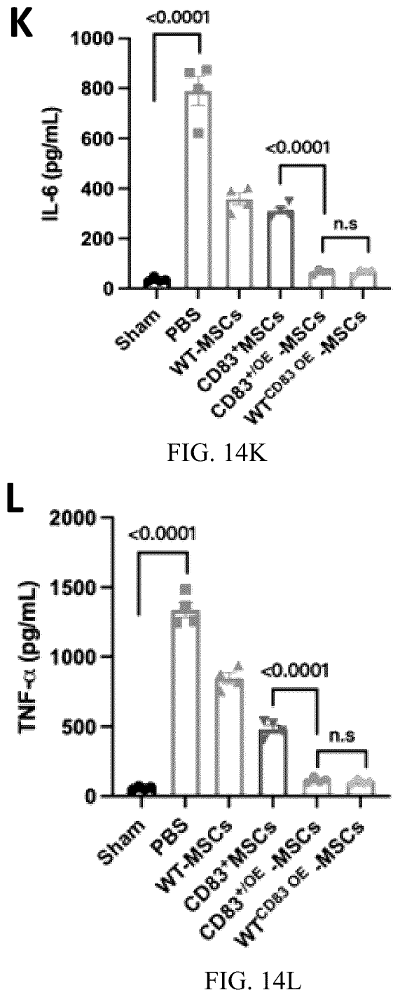

FIGS. 14A-14L illustrate the results of efficacy assessment of WT-MSCs, CD83⁺-MSCs, CD83^{+/OE}-MSCs, and WT^{CD83 OE}-MSCs treated with IFN-γ and TNF-α combined with anti-inflammatory empowerment in the treatment of CLP sepsis mice. FIG. 14A shows the comparative results of the survival rates of CLP sepsis mice treated with the sham operation control (sham), PBS control group, WT-MSCs, CD83⁺-MSCs, CD83^{+/OE}-MSCs, and WT^{CD83 OE}-MSCs groups after 168 h. The results show that the four groups of anti-inflammatory and empowered MSCs can significantly enhance survival rate of the CLP sepsis mice. In comparison, the two groups of MSCs (CD83 overexpressed CD83^{+/OE}-MSCs and WT^{CD83 OE}-MSCs) have the best protective effect on the survival of mice, and there is no difference between the two groups. Further, the protective effect of the CD83⁺-MSCs group on the survival of CLP sepsis mice is significantly better than that of the WT-MSCs group.

### 3. Protective Effect of Immunosuppressive or Anti-Inflammatory Function-Prone CD83⁺ Human Umbilical Cord MSCs on Organ Function in CLP Sepsis Mice

FIGS. 14B-14I show the improvement effect of sham operation control (sham), PBS control group, WT-MSCs, CD83⁺-MSCs, CD83^{+/OE}-MSCs, and WT^{CD83 OE}-MSCs groups on the hepatic function (including glutamate aminotransferase AST and glutamate aminotransferase ALT), renal function (including serum creatinine, blood urea nitrogen, and amylase AMS), and cardiac function (including lactate dehydrogenase LDH, α-hydroxybutyrate dehydrogenase α-HBDH, and creatine kinase CK) after 72 h treatment of CLP sepsis mice. The results show that liver, kidney, and cardiac functions are severely impaired in CLP sepsis mice in the PBS control group and the above indexes are significantly increased as compared to the negative control. After treatment with the above four kinds of anti-inflammatory function-prone MSCs, the above indexes reflecting liver, kidney, and cardiac functions are restored to varying degrees. In comparison, the two groups of MSCs (CD83 overexpressed CD83^{+/OE}-MSCs and WT^{CD83 OE}-MSCs) have the best improvement effects on liver, kidney and cardiac functions of mice, and there is no difference between the two kinds of MSCs. Secondly, the improvement effect of CD83⁺-MSCs group on liver, kidney, and cardiac functions of CLP sepsis mice is significantly better than that of the WT-MSCs group.

### 4. Anti-Inflammatory Effect of Immunosuppressive or Anti-Inflammatory Function-Prone CD83⁺ Human Umbilical Cord MSCs on CLP Sepsis Mice

FIGS. 14J-14L show the anti-inflammatory effects of sham operation control (sham), PBS control group, WT-MSCs, CD83⁺-MSCs, CD83^{+/OE}-MSCs, and WT^{CD83 OE}-MSCs on CLP sepsis mice after the treatment of 72 h, respectively. The results show that compared with the negative control, the serum inflammatory indicators IL-1B, IL-6, and TNF-α are significantly increased in CLP sepsis mice (PBS control group), while the serum levels of IL-1B, IL-6 and TNF-α are significantly decreased after treatment with the above four anti-inflammation empowered MSCs. In comparison, the two groups of MSCs (CD83 overexpressed CD83^{+/OE}-MSCs and WT^{CD83 OE}-MSCs) have the best inhibitory effect on the above three inflammatory factors, and there is no difference between the two kinds of MSCs. In terms of inhibition of inflammatory factor expression, the CD83⁺-MSCs group is slightly better than the WT-MSCs group.

### 5. Protective Effect of Immunosuppressive or Anti-Inflammatory Function-Prone CD83⁺ Human Umbilical Cord MSCs on Liver and Lung Tissue Injury in CLP Sepsis Mice

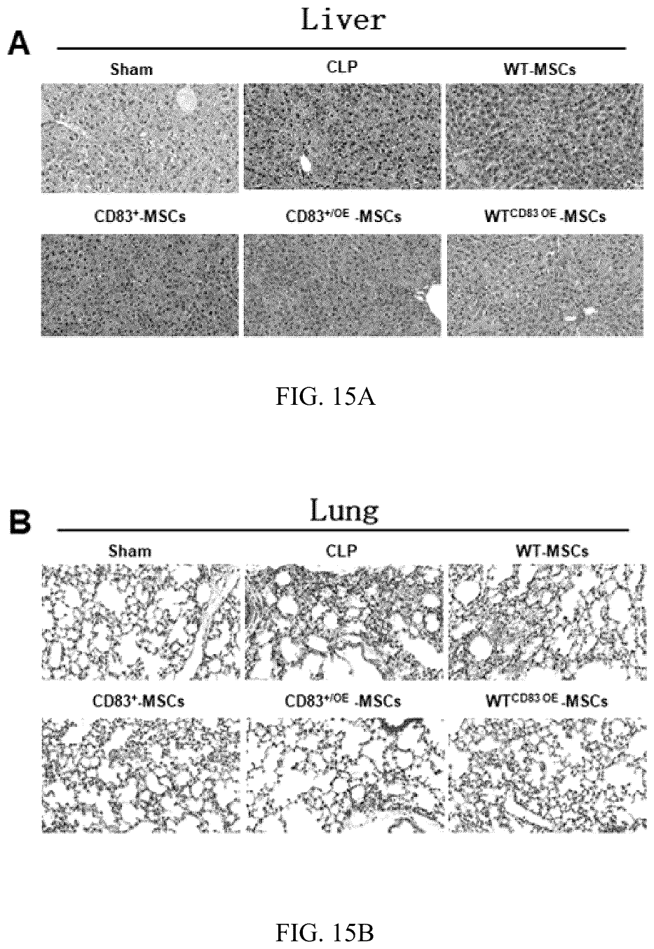

FIGS. 15A-15B show histopathological examination results of hematoxylin and eosin (H&E) staining in CLP sepsis mice treated with sham operation control (sham), PBS control, WT-MSCs, CD83⁺-MSCs, CD83^{+/OE}-MSCs and WT^{CD83 OE}-MSCs for 72 h, respectively. The results show that compared with the negative control, CLP sepsis mice (PBS control group) have structural damage to the liver lobules, with a large amount of inflammatory cell infiltration in the liver parenchyma, thickening of the alveolar walls, infiltration of a large number of inflammatory cells into the lung interstitium and alveolar spaces, and a large amount of exudate in the alveoli. In contrast, after treatment with the above four anti-inflammatory function-prone MSCs, liver and lung structural damage is reduced, and inflammatory cell infiltration in the tissues is reduced. In comparison, the two groups of MSCs (CD83 overexpressed CD83^{+/OE}-MSCs and WT^{CD83 OE}-MSCs) have the best protective effect on liver and lung tissue damage, and there is no difference between the two groups.

The above embodiments are only used to illustrate the technical solutions of the present disclosure, not to limit them. Although the present disclosure has been described in detail with reference to the foregoing embodiments, a person of ordinary skill in the art should understand that it is still possible to make modifications to the technical solutions recorded in the foregoing embodiments, or to make equivalent replacements for some or all of the technical features therein; and such modifications or replacements do not make the corresponding technical solutions fall out of the scope of the technical solutions of the various embodiments of the present disclosure, which shall be covered by the scope of the claims and the present disclosure. These modifications or substitutions do not detach the essence of the corresponding technical solutions from the scope of the technical solutions of the embodiments of the present disclosure, which should be covered by the scope of the claims and disclosure of the present disclosure.

Finally, the above embodiments are only used to illustrate the technical program of the present disclosure rather than limitation, although the present disclosure is described in detail with reference to the better embodiments, the person of ordinary skill in the field should understand that the technical program of the present disclosure can be modified or equivalent replacement without departing from the purpose and scope of the technical program of the present disclosure, which should be covered by the scope of the claims in the present disclosure.

What is claimed is:

1. A method for preventing/treating of an autoimmune disease or an inflammatory-related disease, comprising: administering a therapeutically effective amount of a pharmaceutical composition comprising CD83⁺ mesenchymal stem cells/CD83⁺ overexpressed mesenchymal stem cells to a subject suffering from the autoimmune disease or the inflammatory-related disease.

2. The method of claim 1, wherein the CD83⁺ mesenchymal stem cells/CD83⁺ overexpressed mesenchymal stem cells increase secretion of an anti-inflammatory factor or an immunosuppressive factor.

3. The method of claim 2, wherein the anti-inflammatory factor comprises at least one of IL-10, IDO, PD-L1, GDNF, IL-10Rα, IL-2Rγ, IL-4, IL-4R, IL-17B, LIF, GM-CSFRα, TIMP-3, and IL-2Rβ.

4. The method of claim 2, wherein the immunosuppressive factor comprises at least one of IL-10, IDO, PD-L1, GDNF, IL-10Rα, IL-2Rγ, IL-4, IL-4R, IL-17B, LIF, GM-CSFRα, TIMP-3, and IL-2Rβ.

5. The method of claim 1, wherein the autoimmune disease comprises at least one of rheumatoid arthritis, scleroderma, systemic lupus erythematosus, ankylosing spondylitis, myasthenia gravis, or ulcerative colitis; and the inflammatory-related disease comprises at least one of sepsis, degenerative arthritis, or neonatal bronchopulmonary dysplasia pneumonia.

6. The method of claim 1, wherein the CD83⁺ mesenchymal stem cells/CD83⁺ overexpressed mesenchymal stem cells are obtained by a preparation process comprising:
(a) isolating and culturing mesenchymal stem cells; and
(b) sorting and obtaining immunosuppressive or anti-inflammatory function-prone CD83⁺ mesenchymal stem cells/CD83⁺ overexpressed mesenchymal stem cells by magnetic beads coupled with anti-CD83 antibodies and flow sorting technology, or lentiviral gene transduction technology.

7. The method of claim 6, wherein the mesenchymal stem cells are derived from human adipose, dental pulp, bone marrow, umbilical cord, placenta, or cord blood.

8. The method of claim 1, wherein the pharmaceutical composition further comprises CD83⁺PD-L1⁺ mesenchymal stem cells.

9. The method of claim 8, wherein the CD83⁺PD-L1⁺ mesenchymal stem cells increase the secretion of an anti-inflammatory factor or an immunosuppressive factor.

10. The method of claim 9, wherein the anti-inflammatory factor comprises at least one of IL-10, IDO, PD-L1, GDNF, IL-10Rα, IL-2Rγ, IL-4, IL-4R, IL-17B, LIF, GM-CSFRα, TIMP-3, and IL-2Rβ.

11. The method of claim 9, wherein the immunosuppressive factor comprises at least one of IL-10, IDO, PD-L1, GDNF, IL-10Rα, IL-2Rγ, IL-4, IL-4R, IL-17B, LIF, GM-CSFRα, TIMP-3, and IL-2Rβ.

12. The method of claim 8, wherein the autoimmune disease comprises at least one of rheumatoid arthritis, scleroderma, systemic lupus erythematosus, ankylosing spondylitis, myasthenia gravis, or ulcerative colitis; and the inflammatory-related disease comprises at least one of sepsis, degenerative arthritis, or neonatal bronchopulmonary dysplasia pneumonia.

13. A preparation method for CD83⁺PD-L1⁺ mesenchymal stem cells, comprising: obtaining immunosuppressive or anti-inflammatory function enhanced CD83⁺PD-L1⁺ mesenchymal stem cells by inducing and culturing the CD83⁺ mesenchymal stem cells of claim 1 using an immune factor inducing complex.

14. The preparation method for CD83⁺PD-L1⁺ mesenchymal stem cells of claim 13, wherein the immune factor inducing complex comprises an IFN-γ immune factor and a TNF-α immune factor, a concentration of the IFN-γ immune factor is within a range of 5-20 ng/ml, and a concentration of the TNF-α immune factor is within a range of 0-20 ng/mL.

15. The preparation method for CD83⁺PD-L1⁺ mesenchymal stem cells of claim 14, wherein the immune factor inducing complex comprises an IFN-γ immune factor and a TNF-α immune factor, a concentration of the IFN-γ immune factor is 20 ng/ml, and a concentration of the TNF-α immune factor is within a range of 5-20 ng/mL.

16. The preparation method for CD83⁺PD-L1⁺ mesenchymal stem cells of claim 14, wherein the immune factor inducing complex further comprises an albumin excipient.

17. A quality assessment method for the CD83⁺PD-L1⁺ mesenchymal stem cells obtained by the preparation method of claim 13, wherein a positive expression rate of CD83 and a positive expression rate of a PD-L1 receptor in the CD83⁺PD-L1⁺ mesenchymal stem cells are used as quality control indicators of the CD83⁺PD-L1⁺ mesenchymal stem cells.

18. The quality assessment method of claim 17, wherein the positive expression rate of the CD83 is at least greater than or equal to 70%, and the positive expression rate of the PD-L1 receptors is greater than or equal to 30%.

19. A kit, wherein the kit is effectively used for evaluating a quality requirement of the CD83⁺PD-L1⁺ mesenchymal stem cells by detecting the positive expression rate of the CD83 and the positive expression rate of the PD-L1 receptor with the quality assessment method of claim 17; wherein the kit further comprises the immune factor inducing complex.
